(19) United States
(12) Patent Application Publication
(10) Pub. No.: US 2019/0133995 A1
(43) Pub. Date: May 9, 2019

(54) METHODS FOR THE TREATMENT OF INFECTION

(71) Applicant: Corbus Pharmaceuticals, Inc., Norwood, MA (US)

(72) Inventors: Mark A. TEPPER, Newton, MA (US); Derek W. GILROY, London (GB); Madhur MOTWANI, Amravati (IN); Tracey L. BONFIELD, Chesterland, OH (US)

(21) Appl. No.: 16/096,550

(22) PCT Filed: Apr. 28, 2017
(86) PCT No.: PCT/US2017/030236

§ 371 Date: Oct. 25, 2018

Related U.S. Application Data
(60) Provisional application No. 62/329,514, filed on Apr. 29, 2016.
(60) Provisional application No. 62/400,503, filed on Sep. 27, 2016.
(60) Provisional application No. 62/470,419, filed on Mar. 13, 2017.

(51) Int. Cl. A61K 31/353; A61K 31/145; A61K 9/20; A61K 9/48; A61P 31/04; A61P 31/12; A61P 31/10

(52) U.S. Cl. CPC A61K 31/353; A61K 31/145; A61K 9/20; A61P 31/10; A61P 31/04; A61P 31/12; A61K 9/48

(57) ABSTRACT

The invention provides methods for treating an infection in a subject in need thereof by administering to the subject a pharmaceutical composition including ajulemic acid, or a pharmaceutically acceptable salt thereof. In various embodiments, the infection may be a bacterial, viral, or fungal infection. The invention also features methods of treating an infection in a subject in need thereof by administering to the subject ajulemic acid, or a pharmaceutically acceptable salt thereof, and a suitable antibiotic, antifungal, or antiviral.

### BACKGROUND OF THE INVENTION

Tetrahydrocannabinol (THC) is the major psychoactive constituent of marijuana. In addition to mood-altering effects, THC has been reported to exhibit other activities, some of which may have therapeutic value. The potential therapeutic value of THC has led to a search for related compounds which minimize the psychoactive effects, while retaining the activities of potential medicinal value.

One such related synthetic cannabinoid is (6aR,10aR)-1-hydroxy-6,6-dimethyl-3-(2-methyl-2-octanyl)-6a,7,10,10a-tetrahydro-6H-benzo[c]chromene-9-carboxylic acid (also known as ajulemic acid, AJA, JBT-101, Resunab, or Anabasum). Ajulemic acid has been investigated for its potential therapeutic benefits in a number of disease models, including pain, fibrotic diseases, and inflammatory diseases.

The present invention relates to the discovery that ajulemic acid may also be used to treat an infection, such as a bacterial infection, a viral infection, or a fungal infection. Ajulemic acid may be useful for treating an infection where alternative treatments may result in, for example, negative side-effects (e.g., due to chronic use) or an increase in the likelihood of developing resistant pathogens. In particular, ajulemic acid may be useful for the treatment of infection in a patient having an inflammatory disorder, since other known anti-inflammatory agents (e.g., steroid such as prednisone) are known to decrease the ability of a subject to resolve an infection.

### SUMMARY OF THE INVENTION

The present invention provides methods for treating an infection in a subject in need thereof by administering to the subject a pharmaceutical composition including ajulemic acid, or a pharmaceutically acceptable salt thereof. In various embodiments, the infection may be a bacterial, viral, fungal, or other microbial infection. The invention also features methods of treating an infection in a subject in need thereof by administering to the subject ajulemic acid, or a pharmaceutically acceptable salt thereof, and a suitable antibiotic, antifungal, or antiviral.

In a first aspect, the invention features a method of treating an infection in a subject in need thereof. The method includes administering to the subject a pharmaceutical composition including ajulemic acid, or a pharmaceutically acceptable salt thereof, in an amount effective to treat the infection. In some embodiments of this aspect, the subject does not have cystic fibrosis or an HIV infection.

In another aspect, the invention features a method of treating a local infection in a subject in need thereof. The method includes administering to the subject a pharmaceutical composition including ajulemic acid, or a pharmaceutically acceptable salt thereof, in an amount effective to treat said local infection. In some embodiments of this aspect, the subject does not have an HIV infection.

In some embodiments, the local infection is a skin infection, a lung infection, a bronchial infection, a throat infection, an eye infection, an ear infection, a bladder infection, or a urinary tract infection.

In another aspect, the invention features a method of treating a systemic infection in a subject in need thereof. The method includes administering to the subject a pharmaceutical composition including ajulemic acid, or a pharmaceutically acceptable salt thereof, in an amount effective to treat the systemic infection. In some embodiments of this aspect, the subject does not have an HIV infection.

In some embodiments, the infection is a bacterial infection (e.g., a pseudomonas infection, a staphylococcus infection, or streptococcus infection). In some embodiments, administration of the pharmaceutical composition including ajulemic acid reduces the bacterial burden of the infection (e.g., by at least 5%, by at least 10%, by at least 15%, by at least 20%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 95% or more) relative to either pre-treatment levels in the same subject, or relative to a subject having the same type of infection who has not been administered a pharmaceutical composition including ajulemic acid, or a pharmaceutically acceptable salt thereof.

In some embodiments, the infection is a viral infection. In some embodiments, administration of the pharmaceutical composition including ajulemic acid reduces the viral load of the infection (e.g., by at least 5%, by at least 10%, by at least 15%, by at least 20%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 95% or more) relative to either pre-treatment levels in the same subject, or relative to a subject having the same type of infection who has not been administered a pharmaceutical composition including ajulemic acid, or a pharmaceutically acceptable salt thereof.

In some embodiments, the infection is a fungal infection. In some embodiments, administration of the pharmaceutical composition including ajulemic acid reduces the fungal load of the infection (e.g., by at least 5%, by at least 10%, by at least 15%, by at least 20%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 95% or more) relative to either pre-treatment levels in the same subject, or relative to a subject having the same type of infection who has not been administered a pharmaceutical composition including ajulemic acid, or a pharmaceutically acceptable salt thereof.

In another aspect, the invention features, a method of treating a bacterial infection in a subject in need thereof. The method includes administering to the subject a pharmaceutical composition including ajulemic acid, or a pharmaceutically acceptable salt thereof, in an amount effective to treat the bacterial infection.

In another aspect, the invention features, a method of treating a viral infection in a subject in need thereof. The method includes administering to the subject a pharmaceutical composition including ajulemic acid, or a pharmaceutically acceptable salt thereof, in an amount effective to treat the viral infection.

In another aspect, the invention features a method of treating a fungal infection in a subject in need thereof. The method includes administering to the subject a pharmaceutical composition including ajulemic acid, or a pharmaceutically acceptable salt thereof, in an amount effective to treat the fungal infection.

In another aspect, the invention features a method of treating a bacterial infection in a subject in need thereof by combination therapy with ajulemic acid and a suitable antibiotic. The method includes the steps of:
(a) administering a pharmaceutical composition including an antibiotic, or a pharmaceutically acceptable salt thereof; and
(b) administering a pharmaceutical composition including ajulemic acid, or a pharmaceutically acceptable salt thereof;
   wherein the length of time associated with resolution of the bacterial infection is less than the length of time associated with resolution of a bacterial infection of the same type in a subject who has been administered the pharmaceutical composition that includes the antibiotic alone.

In another aspect, the invention features a method of treating a viral infection in a subject in need thereof by combination therapy with ajulemic acid and a suitable antiviral. The method includes the steps of:
(c) administering a pharmaceutical composition including an antiviral, or a pharmaceutically acceptable salt thereof; and
(d) administering a pharmaceutical composition including ajulemic acid, or a pharmaceutically acceptable salt thereof;
   wherein the length of time associated with resolution of the viral infection is less than the length of time associated with resolution of a viral infection of the same type in a subject who has been administered the pharmaceutical composition that includes the antiviral alone.

In another aspect, the invention features a method of treating a fungal infection in a subject in need thereof by combination therapy with ajulemic acid and a suitable antifungal. The method includes the steps of:
(a) administering a pharmaceutical composition comprising an antifungal, or a pharmaceutically acceptable salt thereof; and
(b) administering a pharmaceutical composition comprising ajulemic acid, or a pharmaceutically acceptable salt thereof;
   wherein the length of time associated with resolution of the fungal infection is less than the length of time associated with resolution of a fungal infection of the same type in a subject who has been administered the pharmaceutical composition that includes the antifungal alone.

In some embodiments, the pharmaceutical composition having the antibiotic, antiviral, or antifungal is administered for a period of time before the administration of the pharmaceutical composition having ajulemic acid. In these embodiments, step (a) is performed for a first period of time, step (b) is performed for a second period of time, and step (a) precedes step (b).

In some embodiments, the pharmaceutical composition having ajulemic acid is administered for a period of time before the administration of the pharmaceutical composition having the antibiotic, antiviral, or antifungal. In these embodiments, step (b) is performed for a first period of time, step (a) is performed for a second period of time, and step (b) precedes step (a).

In some embodiments, the pharmaceutical composition having ajulemic acid is administered concurrently with the pharmaceutical composition having the antibiotic, antiviral, or antifungal. In these embodiments step (a) is performed for a first period of time, step (b) is performed for a second period of time, and the first period of time and the second period of time occur concurrently.

In another aspect, the invention features a method of treating a bacterial infection in a subject in need thereof. The method includes administering to the subject a pharmaceutical composition including an antibiotic, or a pharmaceutically acceptable salt thereof, and ajulemic acid, or a pharmaceutically acceptable salt thereof, in an amount effective to treat the bacterial infection.

In another aspect, the invention features a method of treating a viral infection in a subject in need thereof. The method includes administering to the subject a pharmaceutical composition including an antiviral, or a pharmaceutically acceptable salt thereof, and ajulemic acid, or a pharmaceutically acceptable salt thereof, in an amount effective to treat the viral infection.

In another aspect, the invention features a method of treating a fungal infection in a subject in need thereof. The method includes administering to the subject a pharmaceutical composition including an antifungal, or a pharmaceutically acceptable salt thereof, and ajulemic acid, or a pharmaceutically acceptable salt thereof, in an amount effective to treat the fungal infection.

In another aspect, the invention features a method of treating an infection in a subject in need thereof. The method includes the steps of (a) administering to the subject a pharmaceutical composition including ajulemic acid, or a pharmaceutically acceptable salt thereof, in an amount effective to treat the infection, wherein the pharmaceutical composition is administered for a period of time (e.g., 1 day, 2 days, 3 days, 4 days. 5 days, 6 days, 1 week, 2 weeks, 3 weeks, 4 weeks, or more) required to resolve the infection; and (b) discontinuing administration of the pharmaceutical composition for a period of time (e.g., 1 day, 1 week, 2 weeks, 3 weeks, 1 month, 2 months, 3 months, 4 months, 5 months, 6 months, 1 year, or more) following resolution of the infection.

In some embodiments of any of the foregoing aspects, the length of time associated with resolution of the infection is decreased by 20% or more (e.g., 30% or more, 40% or more, 50% or more, 60% or more, 70% or more, 80% or more, or 90% or more), as compared to an infection of the same type in a subject who has not been administered the pharmaceutical composition including ajulemic acid.

In some embodiments of any of the foregoing aspects, the pharmaceutical composition including ajulemic acid is administered orally (e.g., as a capsule or a tablet), by inhalation (e.g, as an aerosol or spray), topically (e.g., as a gel or cream), intravenously, interstitially, via a patch, via an implant, or by ophthalmic administration.

In some embodiments of any of the foregoing aspects, the effective amount of ajulemic acid comprises a dose of about 5 mg per day or less, of about 10 mg per day, of about 20 mg per day, of about 30 mg per day, of about 40 mg per day, or of about 80 mg per day or more. The daily dose may be administered as one dose, two doses, three doses, or more.

In some embodiments of any of the foregoing aspects, the subject is a mammal (e.g., a human, a cat, a dog, a horse, or a pig). Most preferably the subject is a human subject.

In some embodiments of any of the foregoing aspects, the subject has a disease which is associated with or results in an increased occurrence or severity of infections.

In some embodiments of any of the foregoing aspects, the subject has cystic fibrosis.

In some embodiments of any of the foregoing aspects, the subject does not have cystic fibrosis. In some embodiments of any of the foregoing aspects, the subject does not have cystic fibrosis, but has another disease which is associated with or results in an increased occurrence or severity of infections.

In some embodiments of any of the foregoing aspects, the subject does not have an HIV infection.

In some embodiments of any of the foregoing aspects, the subject does not have any other disease or pathology other than the infection.

In particular embodiments of any of the above aspects, the method includes treating a bacterial infection in the subject. The bacterial infection to be treated can be selected from community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, intravascular infection, complicated skin and skin structure infection, complicated intra-abdominal infection, hospital acquired pneumonia, ventilator associated pneumonia, pseudomembranous colitis, enterocolitis, infections associated with prosthetics or dialysis, and any other infection described herein.

In particular embodiments of any of the above aspects, the method includes treating a fungal infection in the subject. The fungal infection to be treated can be selected from a blood stream infection, tissue infection (e.g., lung, kidney, or liver infection) in the subject, or any other type of fungal infection described herein. The fungal infection being treated can be an infection selected from tinea capitis, tinea corporis, tinea pedis, onychomycosis, perionychomycosis, pityriasis versicolor, oral thrush, vaginal candidosis, respiratory tract candidosis, biliary candidosis, eosophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, sporotrichosis, fungal sinusitis, or chronic sinusitis.

### BRIEF DESCRIPTION OF THE FIGURES

FIG. 1 is a graph showing the bacterial load (measured as CFUs per ml) of Pseudomonas aeruginosa in wild-type (C57BL/6J) mice treated with vehicle, 1 mg/kg AJA, or 5 mg/kg AJA for 10 days. The 5 mg/kg dose was effective at decreasing the overall number of bacterial CFUs in the lungs.

FIG. 2 is a graph depicting the change in body weight in Pseudomonas infected cystic fibrosis (CF) and WT mice following treatment with ajulemic acid (+ AJA), as compared to mice not treated with placebo (− AJA).

FIG. 3 is a schematic depicting, in brief, a study protocol for determining the effect of AJA treatment on Pseudomonas infection in the lungs of WT and CF models of infection.

FIG. 4 is a graph depicting the change in bronchial leukocytes in total bronchoalveolar lavage (BAL) and lungs, combined, of Pseudomonas infected CF and WT mice following treatment with ajulemic acid.

FIG. 5 is a graph depicting the change in white blood cells in lungs of Pseudomonas infected CF and WT mice following treatment with ajulemic acid (AJ), as compared to mice not treated with placebo (Dil).

FIG. 6 is a graph depicting the change in neutrophil counts in lungs of Pseudomonas infected CF and WT mice following treatment with ajulemic acid (AJ), as compared to mice not treated with placebo (Dil).

FIG. 7 is a graph depicting the change in the relative number of alveolar macrophages in lungs of Pseudomonas infected CF and WT mice following treatment with ajulemic acid.

FIG. 8 is a graph depicting the change in the bacterial count in lungs of Pseudomonas infected CF and WT mice following treatment with ajulemic acid.

FIG. 9 is a series of images depicting the effects of ajulemic acid on vascular blood flow at time of inflammatory onset (4 hr) (vascular hyper-reactivity/local blood flow).

FIG. 10 is a series of images depicting the effects of ajulemic acid on vascular blood flow at 24 and 48 hours after inflammatory onset, (vascular hyper-reactivity/local blood flow), as compared to placebo.

FIG. 11 is a graph depicting the time course of effects of ajulemic acid on vascular blood flow after inflammatory onset (vascular hyper-reactivity/local blood flow).

FIG. 12 is a set of graphs that depict the effect of ajulemic acid (5 mg or 20 mg) on neutrophil levels in the blister model.

FIG. 13 is a graph that depict the time course of the effect of ajulemic acid (20 mg) on neutrophil levels in the blister model.

FIG. 14 is a graph that depicts the effect of ajulemic acid (5 mg or 20 mg) treatment on macrophage numbers at 10 hours after injection of UV-killed E. coli (UVKEc) in the blister model.

FIG. 15 is a series of graphs showing that treatment with ajulemic acid (5 mg or 20 mg) increases pro-resolving macrophages during the resolution arm of an infection-induced innate immune response in humans.

FIG. 16 is a series of graphs showing the effects of ajulemic acid (5 mg or 20 mg) treatment of IL-8 cytokine levels at 4 hr and 10 hr after injection of UVKEc in the blister model.

FIG. 17 is a series of graphs showing the effects of ajulemic acid (5 mg or 20 mg) treatment on endotoxin levels, wherein decreases endotoxin is indicative of increased bacterial clearance at 4 hr and 10 hr after injection of UVKEc in the blister model.

FIG. 18 is a graph depicting the time course effect of ajulemic acid on C-reactive protein levels in the blister model.

FIG. 19 is a graph showing that treatment with ajulemic acid is associated with a dose-dependent reduction in acute pulmonary exacerbations requiring administration of intravenous antibiotics in subjects having cystic fibrosis.

FIG. 20 is a graph showing that treatment with ajulemic acid is associated with a dose-dependent reduction in acute pulmonary exacerbations treated with any new antibiotic in subjects having cystic fibrosis.

### DEFINITIONS

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “about” refers to a value that is within 10% above or below the value being described.

As used here, any values provided in a range of values include both the upper and lower bounds, and any values contained within the upper and lower bounds.

As used herein, the term “treat” or “treatment” includes administration of a compound, e.g., by any route, e.g., orally, topically, or by inhalation to a subject. The compound can be administered alone or in combination with one or more additional compounds. Treatments may be sequential, with the present compound being administered before or after the administration of other agents. Alternatively, compounds may be administered concurrently. The subject, e.g., a patient, can be one having a disorder (e.g., a disorder as described herein), a symptom of a disorder, or a predisposition toward a disorder. Treatment is not limited to curing or complete healing, but can result in one or more of alleviating, relieving, altering, partially remedying, ameliorating, improving or affecting the disorder, reducing one or more symptoms of the disorder or the predisposition toward the disorder. In an embodiment the treatment (at least partially) alleviates or relieves symptoms related to infection. In some embodiments, the treatment decreases the length of time associated with resolution of the infection by 20% or more (e.g., 30% or more, 40% or more, 50% or more, 60% or more, 70% or more, 80% or more, or 90% or more), as compared to an infection of the same type in a subject who has not been administered the treatment. In some embodiments, the treatment decreases the bacterial burden, fungal load, or the viral load of the infection. In one embodiment, the treatment reduces at least one symptom of the disorder or delays onset of at least one symptom of the disorder. The effect is beyond what is seen in the absence of treatment.

The term “pharmaceutically acceptable salts,” as used herein, refers to salts of compounds of the present invention which possess the desired pharmacological activity, e.g., biological activity, pharmacokinetic activity. Such salts may include acid addition salts formed with inorganic acids such as hydrochloric acid, hydrobromic acid, sulfuric acid, nitric acid, phosphoric acid, among others.

Pharmaceutically acceptable salts also may include base addition salts which may be formed when acidic protons present are capable of reacting with inorganic or organic bases. Acceptable inorganic bases may include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine. Suitable pharmaceutically-acceptable metallic salts include salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. It should be recognized that the particular anion or cation forming a part of any salt of this invention is not critical, so long as the salt, as a whole, is pharmacologically acceptable.

The term “pharmaceutical composition” refers to the combination of an active agent with an excipient, inert or active, making the composition especially suitable for diagnostic or therapeutic use in vivo or ex vivo. A “pharmaceutically acceptable excipient,” after administered to or upon a subject, does not cause undesirable physiological effects. The excipient in the pharmaceutical composition must be “acceptable” also in the sense that it is compatible with the active ingredient and can be capable of stabilizing it. One or more solubilizing agents can be utilized as pharmaceutical excipients for delivery of an active compound. Examples of a pharmaceutically acceptable excipients include, but are not limited to, biocompatible vehicles, adjuvants, additives, and diluents to achieve a composition usable as a dosage form. Examples of other excipients include colloidal silicon oxide, magnesium stearate, cellulose, and sodium lauryl sulfate.

As used herein, the term “carrier” refers to a diluent, adjuvant, excipient, or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, sodium stearate, glycerol monostearate, talc, sodium chloride, glycerol, propylene glycol, water, and ethanol. The present compositions, if desired, can also contain minor amounts of wetting or emulsifying agents, or pH buffering agents.

As used herein, “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents. In some embodiments, a therapeutically effective amount, when administered to a subject in need, will alleviate at least some of the symptoms of infection.

### DETAILED DESCRIPTION OF THE INVENTION

The present invention provides methods for treating an infection in a subject in need thereof by administering to the subject a pharmaceutical composition including ajulemic acid, or a pharmaceutically acceptable salt thereof. In various embodiments, the infection may be a bacterial, viral, or fungal infection. The invention also features methods of treating an infection in a subject in need thereof by administering to the subject ajulemic acid, or a pharmaceutically acceptable salt thereof, and a suitable antibiotic, antifungal, or antiviral.

### Ajulemic Acid

(6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid, AJA, JBT-101, Resunab, or Anabasum) is a synthetic cannabinoid that is structurally related to THC, but which lacks the undesirable psychotropic effects associated with THC. As a result, ajulemic acid has been investigated for its potential therapeutic utility in a number of diseases including fibrotic diseases and inflammatory diseases.

Ajulemic acid has the following structure:

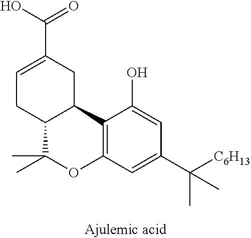

### Therapy

The treatment regimens and pharmaceutical compositions described herein can be used to treat an infection (e.g., a bacterial infection, a viral infection, a fungal infection, a helmintic infection, or a protozoal infection, or another microbial infection).

### Treating an Infection

The invention features a method of treating an infection in a subject in need thereof. The method includes administering to the subject a pharmaceutical composition including ajulemic acid, or a pharmaceutically acceptable salt thereof, in an amount effective to treat the infection. In some embodiments, the subject does not have cystic fibrosis or an HIV infection.

As used herein, the term “treating an infection” refers to a therapeutic treatment of an infection in a subject. A therapeutic treatment slows the progression of the infection, improves the subject's outcome, and/or eliminates the infection. In some embodiments, treating an infection by administering a pharmaceutical composition including ajulemic acid reduces the bacterial burden, viral load, or fungal load of the infection (e.g., by at least 5%, by at least 10%, by at least 15%, by at least 20%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 95% or more) relative to either pre-treatment levels in the same subject, or relative to a subject having the same type of infection who has not been administered a pharmaceutical composition including ajulemic acid, or a pharmaceutically acceptable salt thereof. In some embodiments, treating an infection by administering a pharmaceutical compositing including ajulemic acid reduced the length of time associated with resolution of the infection by 20% or more (e.g., 30% or more, 35% or more, 40% or more, 45% or more, 50% or more, 55% or more, 60% or more, 65% or more, 70% or more, 75% or more, 80% or more, 95% or more, or 90% or more), as compared to an infection of the same type in a subject who has not been administered the pharmaceutical composition including ajulemic acid.

As used herein, the term “infection” refers to the invasion of a subject's cells, tissues, and/or organs by a pathogen, such as bacteria, viruses, fungi, helminths, or protozoans. In some embodiments, the pathogen may grow, multiply, and/or produce toxins in the subject's cells, tissues, and/or organs. In some embodiments, the subject may develop a negative reaction (i.e., an allergic reaction or an immune response) to the pathogen. Examples of infections include, but are not limited to, a bacterial infection, a viral infection, a fungal infection, a helmintic infection, and a protozoal infection.

As used herein, the term “bacterial infection” refers to an infection caused by one or more bacteria. Examples of infection-causing bacteria are well-known in the art and include, but are not limited to, bacteria in the genus Pseudomonas (e.g., Pseudomonas aeruginosa), bacteria of the genus Staphylococcus (e.g., Staphylococcus aureus), bacteria in the genus Streptococcus (e.g., Streptococcus pyogenes), bacteria in the genus Escherichia (e.g., Escherichia coli), bacteria in the genus Vibrio (e.g., Vibrio cholerae), bacteria in the genus Enteritis (e.g., Enteritis salmonella), and bacteria in the genus Salmonella (e.g., Salmonella typhi).

As used herein, the term “viral infection” refers to an infection caused by one or more viruses. Examples of infection-causing viruses are well-known in the art and include, but are not limited to, viruses in the family Retroviridae (e.g., human immunodeficiency virus (HIV)), viruses in the family Adenoviridae (e.g., adenovirus), viruses in the family Herpesviridae (e.g., herpes simplex virus types 1 and 2), viruses in the family Papillomaviridae (e.g., human papillomavirus (HPV)), viruses in the family Poxviridae (e.g., smallpox), viruses in the family Picornaviridae (e.g., hepatitis A virus, poliovirus, rhinovirus), viruses in the family Hepadnaviridae (e.g., hepatitis B virus), viruses in the family Flaviviridae virus (e.g., hepatitus C virus, yellow fever virus, West Nile virus), viruses in the family Togaviridae (e.g., rubella virus), viruses in the family Orthomyxoviridae (e.g., influenza virus), viruses in the family Filoviridae (e.g., ebola virus, marburg virus), and viruses in the family Paramyxoviridae (e.g., measles virus, mumps virus).

As used herein, the term “fungal infection” refers to an infection caused one or more fungi. Examples of infection-causing fungi are well-known in the art and include, but are not limited to, fungi in the genus Aspergillus (e.g., Aspergillus fumigatus, A. flavus, A. terreus, A. niger, A. candidus, A. clavatus, A. ochraceus), fungi in the genus Candida (e.g., Candida albicans, C. parapsilosis, C. glabrata, C. guilliermondii, C. krusei, C. lusitaniae, C. tropicalis), fungi in the genus Cryptococcus (e.g., Cryptococcus neoformans), and fungi in the genus Fusarium (e.g., Fusarium solani, F. verticillioides, F. oxysporum).

As used herein, the term “helmintic infection” refers to an infection caused by one or more helminths. Examples of helminths include, but are not limited to, tapeworms (cestodes), roundworms (nematodes), flukes (trematodes), and monogeneans.

As used herein, the term “protozoal infection” refers to an infection caused by one or more protozoans. Examples of protozoans include, but are not limited to, protozoans in the genus Entamoeba (e.g., Entamoeba histolytica), protozoans in the genus Plasmodium (e.g., Plasmodium falciparum, P. malariae), protozoans in the genus Giardia (e.g., Giardia lamblia), and protozoans in the genus Trypanosoma (e.g., Trypanosoma brucei).

### Local Infection

In some embodiments of the invention, the infection is a local infection. The invention features a method of treating a local infection in a subject in need thereof. The method includes administering to the subject a pharmaceutical composition including ajulemic acid, or a pharmaceutically acceptable salt thereof, in an amount effective to treat the local infection.

In some embodiments, the local infection is a skin infection, a lung infection, a bronchial infection, a throat infection, an eye infection, an ear infection, a bladder infection, or a urinary tract infection.

In some embodiments, the local infection is a mild infection.

In some embodiments, administration of ajulemic acid is associated with a decrease in adverse events and/or a decrease in the occurrence of resistant pathogens relative to other available treatments (e.g., antibiotic treatment).

In some embodiments, the local infection is in a subject having cystic fibrosis (e.g., an infection, for example a pseudomonas infection, in the lungs of a subject having cystic fibrosis).

In some embodiments, the local infection is in a subject who does not have cystic fibrosis.

### Systemic Infection

In some embodiments of the invention, the infection is a systemic infection. The invention features a method of treating a systemic infection in a subject in need thereof. The method includes administering to the subject a pharmaceutical composition including ajulemic acid, or a pharmaceutically acceptable salt thereof, in an amount effective to treat the systemic infection.

In some embodiments, the systemic infection is a chronic infection.

In some embodiments, administration of ajulemic acid is associated with a decrease in adverse events and/or a decrease in the occurrence of resistant pathogens relative to other available treatments (e.g., antibiotic treatment).

### Combination Therapies

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In some embodiments, the invention includes a method of treating an infection (e.g., a bacterial infection, a fungal infection, or a viral infection) in a subject in need thereof by combination therapy with ajulemic acid and a suitable therapeutic (e.g., an antibiotic, an antifungal, or an antiviral therapeutic). The method includes the steps of:
(a) administering a pharmaceutical composition comprising a suitable therapeutic (e.g., an antibiotic, an antifungal, or an antiviral therapeutic), or a pharmaceutically acceptable salt thereof; and
(b) administering a pharmaceutical composition comprising ajulemic acid, or a pharmaceutically acceptable salt thereof;
   wherein the length of time associated with resolution of the infection is less than the length of time associated with resolution of an infection of the same type in a subject who has been administered the pharmaceutical composition that includes the therapeutic of step (a) alone.

Step (a) may be performed for a period of time before step (b), which is also performed for a period of time. Step (b) may be performed for a period of time, after which step (a) is performed for a period of time. Step (a) and step (b) may be performed concurrently.

Wherein the suitable therapeutic (e.g., the antibiotic, antifungal, or antiviral) and the pharmaceutical composition including ajulemic acid are administered during the same period of time, the dosing of each may occur together (either in the same pharmaceutical formulation of separate pharmaceutical formulations) or may occur separately.

### Antibiotic Combination Therapy

In some embodiments of the above-described combination therapies for the treatment of infection in a subject in need thereof, the antibiotic is selected from the group consisting of amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim. The preceding list is meant to be exemplary of antibiotics known to one skilled in the art for the treatment of infection and is not meant to limit the scope of the invention.

### Antifungal Combination Therapy

In some embodiments of the above-described combination therapies for the treatment of infection in a subject in need thereof, the antifungal is selected from the group consisting of amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, triazoles, albaconazole, efinaconazole, epoxiconazole, fluconazole, isavuconazole, itraconazole, posaconazole, propiconazole, ravuconazole, terconazole, voriconazole, thiazoles, abafungin, amorolfin, butenafine, naftifine, terbinafine, anidulafungin, caspofungin, micafungin, ciclopirox, flucytosine, griseofulvin, tolnaftate, and undecylenic acid. The preceding list is meant to be exemplary of antifungals known to one skilled in the art for the treatment of infection and is not meant to limit the scope of the invention.

### Antiviral Combination Therapy

In some embodiments of the above-described combination therapies for the treatment of infection in a subject in need thereof, the antiviral is selected from the group consisting of vidarabine, acyclovir, gancyclovir, valgancyclovir, a nucleoside-analog reverse transcriptase inhibitor (e.g., AZT (Zidovudine), ddl (Didanosine), ddC (Zalcitabine), d4T (Stavudine), or 3TC (Lamivudine)), a non-nucleoside reverse transcriptase inhibitor (e.g., (nevirapine or delavirdine), protease inhibitor (saquinavir, ritonavir, indinavir, or nelfinavir), ribavirin, or interferon). The preceding list is meant to be exemplary of antivirals known to one skilled in the art for the treatment of infection and is not meant to limit the scope of the invention.

### Pharmaceutical Compositions

As described above, the pharmaceutical compositions of the invention additionally include a pharmaceutically acceptable excipient, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The above-described composition, in any of the forms described above, can be used for treating an infection, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, buccally, by ophthalmic administration, or by inhalation. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acids, such as, but not limited to, oleic acid and its glyceride derivatives, are useful in the preparation of injectables, as are natural pharmaceutically acceptable oils, such as, but not limited to, olive oil or castor oil, orpolyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets (e.g. a pressed table), emulsions and aqueous suspensions, dispersions, and solutions. In the case of tablets, commonly used excipients include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable excipient suitable for application to the skin. A “cosmetically acceptable” or “dermatologically-acceptable” composition or component refers a composition or component that is suitable for use in contact with human skin without undue toxicity, incompatibility, instability, or allergic response. The excipient enables an active agent and optional component to be delivered to the skin at an appropriate concentration(s). The excipient thus can act as a diluent, dispersant, solvent, or the like to ensure that the active materials are applied to and distributed evenly over the selected target at an appropriate concentration. The excipient can be solid, semi-solid, or liquid. The excipient can be in the form of a lotion, a cream, or a gel, in particular one that has a sufficient thickness or yield point to prevent the active materials from sedimenting. The excipient can be inert or possess dermatological benefits. It also should be physically and chemically compatible with the active components described herein, and should not unduly impair stability, efficacy, or other use benefits associated with the composition.

### Pharmaceutical Dosage Forms

Various dosage forms of ajulemic acid can be used in the methods of the invention for treating an infection. In some embodiments, the dosage form is an oral dosage form such as a pressed tablet, hard or soft gel capsule, enteric coated tablet, osmotic release capsule, or unique combination of excipients.

In further embodiments, the dosage form includes an additional agent or is provided together with a second dosage form, which includes the additional agent. Exemplary additional agents include an analgesic agent such as an NSAID or opiate, an anti-inflammatory agent or a natural agent such as a triglyceride containing unsaturated fatty acid, or isolated pure fatty acids such as eicosapentaenoic acid (EPA), dihomogamma linolenic acid (DGLA), docosahexaenoic acid (DHA) and others. In additional embodiments, the dosage form includes a capsule wherein the capsule contains a mixture of materials to provide a desired sustained release formulation.

The dosage forms can include a tablet coated with a semipermeable coating. In certain embodiments, the tablet includes two layers, a layer containing ajulemic acid (e.g. ultrapure ajulemic acid) and a second layer referred to as a “push” layer. The semi-permeable coating is used to allow a fluid (e.g., water) to enter the tablet and erode a layer or layers. In certain embodiments, this sustained release dosage form further includes a laser hole drilled in the center of the coated tablet. The ajulemic acid containing layer may include ajulemic acid, a disintegrant, a viscosity enhancing agent, a binding agent, and an osmotic agent. The push layer includes a disintegrant, a binding agent, an osmotic agent, and a viscosity enhancing agent.

The present compositions may be formulated for sustained release (e.g. over a 2 hour period, over a 6 hour period, over a 12 hour period, over a 24 hour period, or over a 48 hour period).

In further embodiments, the dosage form includes a tablet including a biocompatible matrix and ajulemic acid. The sustained release dosage form may also comprise a hard-shell capsule containing bio-polymer microspheres that contains the therapeutically active agent. The biocompatible matrix and bio-polymer microspheres each contain pores for drug release and delivery. These pores are formed by mixing the biocompatible matrix of bio-polymer microsphere with a pore forming agent. Each biocompatible matrix or bio-polymer microsphere is made up of a biocompatible polymer or mixture of biocompatible polymers. The matrix and microspheres can be formed by dissolving the biocompatible polymer and active agent (compound described herein) in a solvent and adding a pore-forming agent (e.g., a volatile salt). Evaporation of the solvent and pore forming agent provides a matrix or microsphere containing the active compound. In additional embodiments, the sustained release dosage form includes a tablet, wherein the tablet contains ajulemic acid and one or more polymers and wherein the tablet can be prepared by compressing the ajulemic acid and one or more polymers. In some embodiments, the one or more polymers may comprise a hygroscopic polymer formulated with ajulemic acid. Upon exposure to moisture, the tablet dissolves and swells. This swelling allows the sustained release dosage form to remain in the upper GI tract. The swelling rate of the polymer mixture can be varied using different grades of polyethylene oxide.

In other embodiments, the sustained release dosage form includes a capsule further including particle cores coated with a suspension of active agent and a binding agent which is subsequently coated with a polymer. The polymer may be a rate-controlling polymer. In general, the delivery rate of the rate-controlling polymer is determined by the rate at which the active agent is dissolved.

In some embodiments, one or more of the therapeutic agents that can be used in the methods of the invention for treating an infection may be formulated with a pharmaceutically acceptable carrier, vehicle or adjuvant. The term “pharmaceutically acceptable carrier, vehicle, or adjuvant” refers to a carrier, vehicle or adjuvant that may be administered to a subject, together with the present compounds, and which does not destroy the pharmacological activity thereof and is nontoxic when administered in doses sufficient to deliver a therapeutic amount of the compound.

Pharmaceutically acceptable carriers, adjuvants and vehicles that may be used in the dosage forms of this invention include, but are not limited to, ion exchangers, alumina, aluminum stearate, lecithin, self-emulsifying drug delivery systems (SEDDS) such as d-E-tocopherol polyethylene-glycol 1000 succinate; surfactants used in pharmaceutical dosage forms such as Tweens or other similar polymeric delivery matrices; serum proteins such as human serum albumin; buffer substances such as phosphates, glycine, sorbic acid, potassium sorbate, partial glyceride mixtures of saturated vegetable fatty acids, water, salts; or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxmethylcellulose, polyacrylates, waxes, polyethylene-polyoxypropylene-block polymers, polyethylene glycol and wool fat. Cyclodextrins such as alpha, beta and .gamma.-cyclodextrin, or chemically modified derivatives such as hydroxyalkylcyclodextrins, including 2- and 3-hydroxypropyl-beta cyclodextrins, or other solubilized derivatives may also be advantageously used to enhance delivery of compounds of the formulae described herein that can be used in the methods of the invention for preventing and/or treating fibrotic conditions. In certain embodiments, unit dosage formulations are compounded for immediate release, though unit dosage formulations compounded for delayed or prolonged release of one or both agents are also disclosed.

In some embodiments, the therapeutic agents that can be used in the present methods are formulated in a single unit dose such that the agents are released from the dosage at different times.

In another embodiment, for example, where one or more of the therapeutic agents is administered once or twice per day, the agent is formulated to provide extended release. For example, the agent is formulated with an enteric coating. In an alternative embodiment, the agent is formulated using a biphasic controlled release delivery system, thereby providing prolonged gastric residence. For example, in some embodiments, the delivery system includes (1) an inner solid particulate phase formed of substantially uniform granules containing a pharmaceutical having a high water solubility, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophobic polymers, one or more hydrophobic polymers and/or one or more hydrophobic materials such as one or more waxes, fatty alcohols and/or fatty acid esters, which may be compressed into tablets or filled into capsules. In some embodiments, the agent is incorporated into polymeric matrices comprised of hydrophilic polymers that swell upon imbibition of water to a size that is large enough to promote retention of the dosage form in the stomach during the fed mode.

The ajulemic acid in the formulation may be formulated as a combination of fast-acting and controlled release forms. For example, the ajulemic acid is formulated with a single release property. For example, it is not present in a modified release form, e.g., a controlled release form.

The present compositions may be taken just prior to or with each of three meals, each of two major meals, or one meal. In other embodiments, a composition disclosed herein can be administered one or more times daily (e.g., once daily, twice daily, or three times daily) and need not be administered just before or with a meal.

The present compounds or compositions may be administered orally, for example as a component in a dosage form. The dosage forms may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The dosage forms of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Non-limiting examples of capsules include but are not limited to gelatin capsules, HPMC, hard shell, soft shell, or any other suitable capsule for holding a sustained release mixture. The solvents used in the above sustained release dosage forms include, but are not limited to ethyl acetate, triacetin, dimethyl sulfoxide (DIV1S0), propylene carbonate, N-methylpyrrolidone (NMP), ethyl alcohol, benzyl alcohol, glycofurol, alpha-tocopherol, Miglyol 810, isopropyl alcohol, diethyl phthalate, polyethylene glycol 400 (PEG 400), triethyl citrate, and benzyl benzoate.

The viscosity modifiers that may be used in the above pharmaceutical compositions include, but are not limited to caprylic/capric triglyceride (Migliol 810), isopropyl myristate (IPM), ethyl oleate, triethyl citrate, dimethyl phthalate, benzyl benzoate and various grades of polyethylene oxide. The high viscosity liquid carrier used in the above sustained release dosage forms include, but are not limited to sucrose acetate isobutyrate (SA1B) and cellulose acetate butyrate (CAB) 381-20.

Non-limiting examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof; ethylene vinyl acetate copolymers, polyethylene, copolymers of ethylene, polyolefins including ethylene oxide copolymers (e.g., Engage®—Dupont Dow Elastomers), polyamides, cellulosic materials, polyurethanes, polyether blocked amides, and copolymers (e.g., PEBAX®, cellulosic acetate butyrate and polyvinyl acetate). Non-limiting examples of disintegrants that may be employed in the above sustained release dosage forms include but are not limited to croscarmellose sodium, crospovidone, sodium alginate or similar excipients.

Non-limiting examples of binding agents that may be employed in the above dosage forms include but are not limited to hydroxyalkylcellulose, a hydroxyalkylalkylcellulose, or a polyvinylpyrrolidone.

Non-limiting examples of osmotic agents that may be employed in the above dosage forms include but are not limited to, sorbitol, mannitol, sodium chloride, or other salts. Non-limiting examples of biocompatible polymers employed in the above sustained release dosage forms include but are not limited to poly(hydroxyl acids), polyanhydrides, polyorthoesters, polyamides, polycarbonates, polyelkylenes, polyelkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly (vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

Non-limiting examples of hygroscopic polymers that may be employed in the above dosage forms include but are not limited to polyethylene oxide (e.g., Polyox® with MWs from 4,000,000 to 10,000,000), cellulose hydroxymethyl cellulose, hydroxyethyl-cellulose, crosslinked polyacrylic acids and xanthum gum.

Non-limiting examples of rate-controlling polymers the may be employed in the above dosage forms include but are not limited to polymeric acrylate, methacrylatelacquer or mixtures thereof, polymeric acrylate lacquer, methacrylate lacquer, an acrylic resin including a copolymer of acrylic and methacrylic acid esters or an ammonium methacrylate lacquer with a plasticizer.

### Kits

A dosage form described herein may be provided in a kit. The kit includes (a) a compound used in a method described herein, and, optionally (b) informational material. The informational material can be descriptive, instructional, marketing or other material that relates to the methods described herein and/or the use of the dosage form for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to use a compound or composition described herein in a suitable manner to perform the methods described herein, e.g., carry out a reaction to produce a compound described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a dosage form described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a dosage form described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the dosage form is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a dosage form described herein.

The containers of the kits can be air tight, waterproof (e.g., impermeable to changes in moisture or evaporation), and/or light-tight.

The kit optionally includes a device suitable for use of the dosage form, e.g., a syringe, pipette, forceps, measured spoon, swab (e.g., a cotton swab or wooden swab), or any such device.

The kits of the invention can include dosage forms of varying strengths to provide a subject with doses suitable for one or more of the initiation phase regimens, induction phase regimens, or maintenance phase regimens described herein. Alternatively, the kit can include a scored tablet to allow the user to administered divided doses, as needed.

The specific compounds and compositions (e.g., a pharmaceutical composition including ajulemic acid, or a pharmaceutically acceptable salt thereof) have been disclosed. A number of embodiments of the invention have been described. Nevertheless, it will be understood that various modifications may be made without departing from the spirit and scope of the invention. Accordingly, other embodiments are within the scope of the following claims. It should be apparent, however, to those skilled in the art that many more modifications besides those already described are possible without departing from the inventive concepts herein. The inventive subject matter, therefore, is not to be restricted except in the spirit of the disclosure.

All patents, patent publications and publications mentioned herein are incorporated herein by reference in their entirety to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Study of Ajulemic Acid in Mice Infected with Pseudomonas aeruginosa Beads in the Lung in WT Mice

### SUMMARY

Ajulemic acid (AJA) was tested in mice inoculated with Pseudomonas aeruginosa to determine its effect on treatment of infection, including the ability to promote bacterial clearance. Mice (WT, C57BL/6J) were inoculated with Pseudomonas aeruginosa agarose beads in the lungs and then treated twice daily with oral doses of ajulemic acid at 1 mg/kg and 5 mg/kg. Ajulemic acid was then administered by gavage at 1 mg/kg or 5 mg/kg dose BID in 2% methylcellulose for 10 days starting 24 hours after establishing chronic Pseudomonas aeruginosa infection. WT animals in this study were followed daily for clinical score and weights for 10 days. At Day 10, animals were euthanized and evaluated for bacteria load (colony forming units, cfus), total bronchoalveolar lavage (BAL), white blood cell counts (WBCs), and differential cell counts. In WT C57BL/6J mice, ajulemic acid was well tolerated and more efficient at treating infection than vehicle.

Methods

The study was conducted with 40 wild type female C57BL/6J mice. Each group of mice was inoculated with 10⁵ colony forming units (CFUs) of Pseudomonas aeruginosa (PAM 5715, a CF clinical isolate). One day post infection, mice were given either 2% methylcellulose, 2% methylcellulose+1 mg/kg ajulemic acid, or 2% methylcellulose+5 mg/kg ajulemic acid BID. Animals were followed for 10 days. At day 10, animals were euthanized for BAL CFUs, differentials, total white blood cell count with fluid, and pellet saved for future studies.

Treatment and control groups include the following Pseudomonas aeruginosa infected controls treated with 2% methycellulose, Pseudomonas aeruginosa infected animals treated with 1 mg/kg BID AJA in 2% methylcellulose, Pseudomonas aeruginosa infected animals treated with 5 mg/kg BID AJA in 2% methylcellulose, and untreated baseline controls.

Results

The white blood cell response was elevated in the ajulemic acid treatment groups (1 mg/kg and 5 mg/kg) at day 3 relative to controls infected or not infected. All groups normalized at day 10, without elevation of white blood cells compared to controls.

Consistent with the white blood cell counts, there were elevated levels of neutrophils in all treatment groups relative to the non-treated control. By day 10, there was a shift in the treatment groups towards an increase in alveolar macrophages and decreased neutrophils. There was no significant difference or levels of lymphocytes or eosinophils in any of the groups.

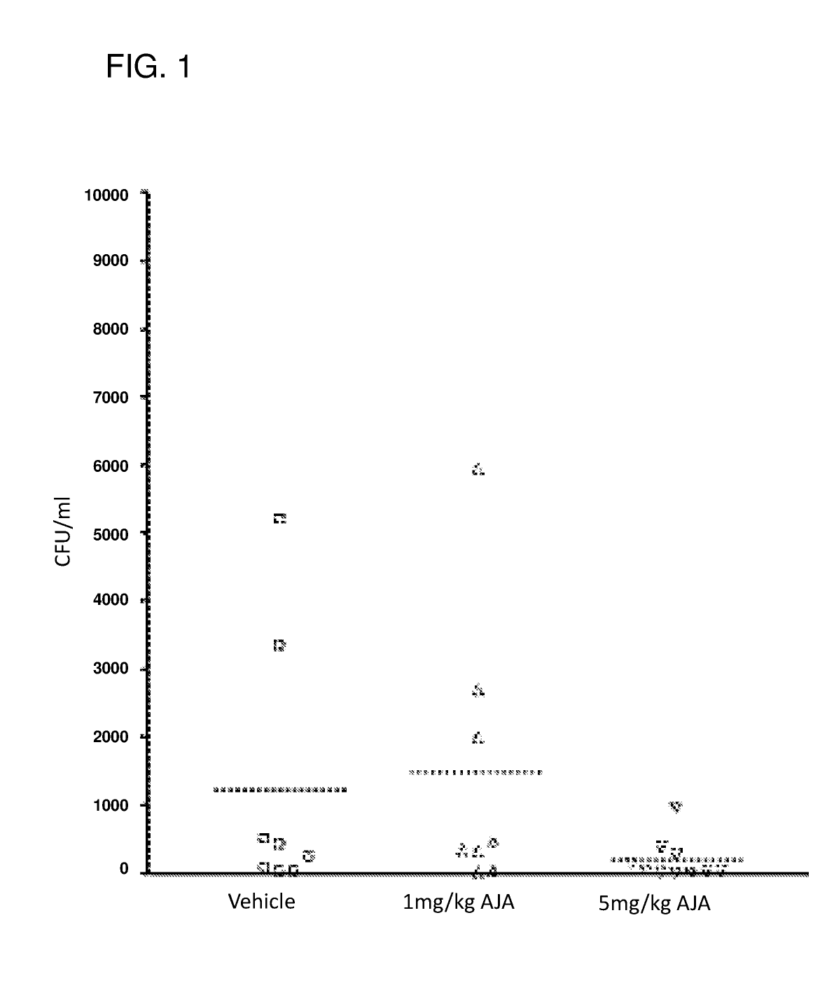

In terms of the impact of the treatment with ajulemic acid on bacterial load of Pseudomonas aeruginosa, at day 10 the 5 mg/kg dose was effective at decreasing the overall number of bacterial CFUs in the lungs (FIG. 1).

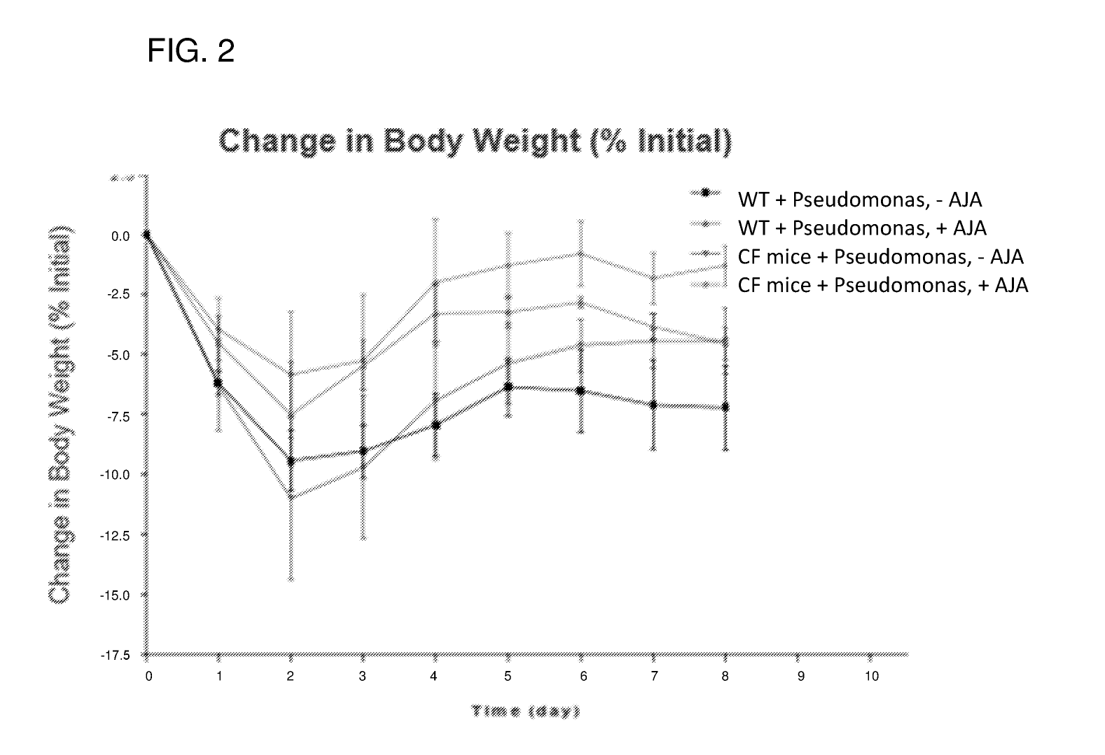

The weight profiles were consistent between the groups with no real adverse effect on how the animals maintained their weight (FIG. 2).

In WT C57BL/6J mice, ajulemic acid was well tolerated and more efficient at treating infection than vehicle.

### Example 2. Study of Ajulemic Acid in Mice Infected with Pseudomonas aeruginosa Beads in the Lung in CFTR KO Mice

Summary

It has previously been established that Cftr deficient animals have a more robust inflammatory response to Pseudomonas aeruginosa infection, and accordingly are very inefficient at resolving the bacterial burden. Further, post-infection Cftr deficient animals loose significant weight and have higher clinical scores. Ajulemic acid (AJA) was tested in Cftr knockout mice inoculated with Pseudomonas to determine its effect on treatment of infection, including its ability to promote bacterial clearance.

A limited number of both WT (C57BL/6J, n=5) and Cftr KO (congenic-homologous, DelF508-FABP gut corrected animals, n=5) were evaluated for safety, toxicity, and efficacy upon oral dosing of 5 mg/kg ajulemic acid BID. As controls, PA infected WT C57BL/6J and Cftr KO mice were given the 2% methylcellulose vehicle. Cftr KO and WT animals in this study were followed daily for clinical score and weights for 10 days. At Day 10, animals were euthanized and evaluated for bacteria load (colony forming units, cfus), total bronchoalveolar lavage (BAL) white blood cell counts (WBCs), and differential cell counts.

This study suggests that ajulemic acid may be effective to treat infection in animals having an increased susceptibility to infection and/or a decreased ability to resolve an infection, for example animals having cystic fibrosis.

Methods

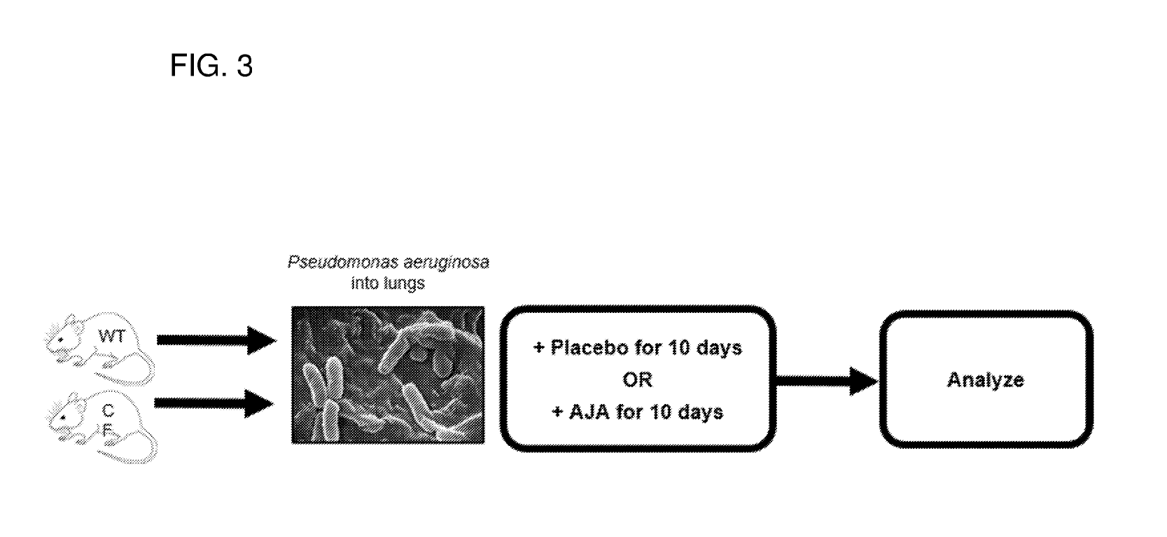

The design of this study is summarized in FIG. 3. Mice were inoculated with Pseudomonas aeruginosa (PA) agaroses beads in the lungs and then treated twice daily with oral doses of ajulemic acid at 1 mg/kg and 5 mg/kg. Ajulemic acid was then administered by gavage at 1 mg/kg or 5 mg/kg dose BID in 2% methylcellulose for 10 days starting 24 hours after establishing chronic Pseudomonas aeruginosa (PA) infection. The study included 4 groups:
1) C57BL/6J+vehicle (WT DIL, n=5),
2) C57BL/6J+5 mg/kg ajulemic acid BID (WT AJ, n=5),
3) Cftr KO+vehicle (CF DIL, n=5), and
4) Cftr KO+5 mg/kg ajulemic acid BID (CF AJ, n=5),
   and all animals were chronically infected with Pseudomonas aeruginosa using a standard agarose bead model.

Study Protocol

The study protocol is summarized in Table 1.

TABLE 1
Pseudomonas aeruginosa agarose bead model study protocol summary

| Day | Action |
|---|---|
| Day −3 | Validated the Pseudomonas aeruginosa culture |
| Day −2 | Second flask of Pseudomonas aeruginosa was started to standardize the growth |
| | kinetics and viability of the bacteria |
| Day −1 | Agarose bead preparation was done incorporating viable 10⁵ Pseudomonas |
| | aeruginosa into agarose beads with a known size and distribution. These beads |
| | were plated to determine success of the procedure prior to giving to the mice. |
| Day 0 | Cultured beads were validated and plotted using CFUs versus titer to determine |
| | the quantitative value of the agarose bead preparation. This process was used |
| | to identify the 10⁵ CFU dose of Pseudomonas aeruginosa. Recorded weights of |
| | animal groups, followed by trans-tracheal administration of agarose bead |
| | preparation. |
| Day +1 | Mice were evaluated again for weights and clinical scores. Start of the |
| | administration of therapeutic dose. |
| Day +2 to 9 | Recorded all weights and clinical scores, and continued therapeutic |
| | administration. |
| Day +10 | After all of the mice are evaluated for weights, clinical scores, animals were |
| | euthanized to evaluate the status of infection and inflammation using |
| | bronchoalveolar lavage (BAL). BAL fluid was evaluated for cellular differential, |
| | bacterial load and elastase with the remaining fluid aliquoted for biomarker |
| | assessment including: TNFα, IL-1β, IFNy, KC, MIP-1α, MIP-1β, MCP-1, IL-6, IL- |
| | 10, IL-17, G-CSF, GM-CSF and calprotectin. BAL fluid and cell pellet were kept |
| | for future analysis by gene array, if deemed reasonable by the study outcome. |
| | BALed lungs were be homogenized for bacterial load, lung homogenate pellets |
| | and supernatants were saved for further analysis. Serum was obtained from all |
| | animals for systemic biomarkers associated with the CF model and aliquots were |
| | saved for future analysis. Bone marrow was also obtained for hematopoietic |
| | effects of the therapeutic in question. |

Results

Ajulemic acid improved survival of Cftr KO (Cftr −/−) animals from 3/5 (vehicle only) to 5/5 (5 mg/kg ajulemic acid BID). The survival rate is summarized in Table 2.

TABLE 2
Survival rate of mice infected with Pseudomonas
aeruginosa beads in the lung

| | Survival Rate |
|---|---|
| Treatment group | (Day 10) |
| WT | 5/5 (100%) |
| WT + AJA | 5/5 (100%) |
| CF | 3/5 (60%) |
| CF + AJA | 5/5 (100%) |

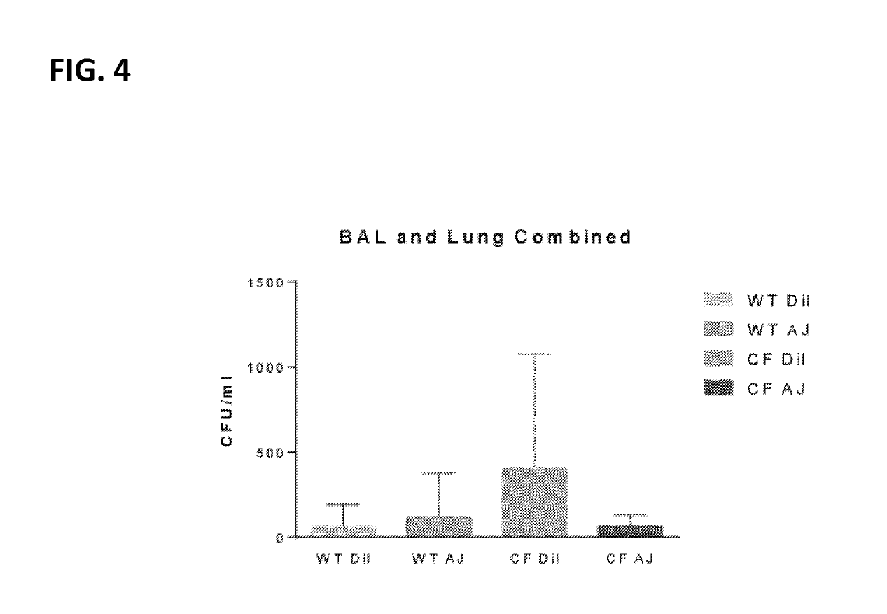

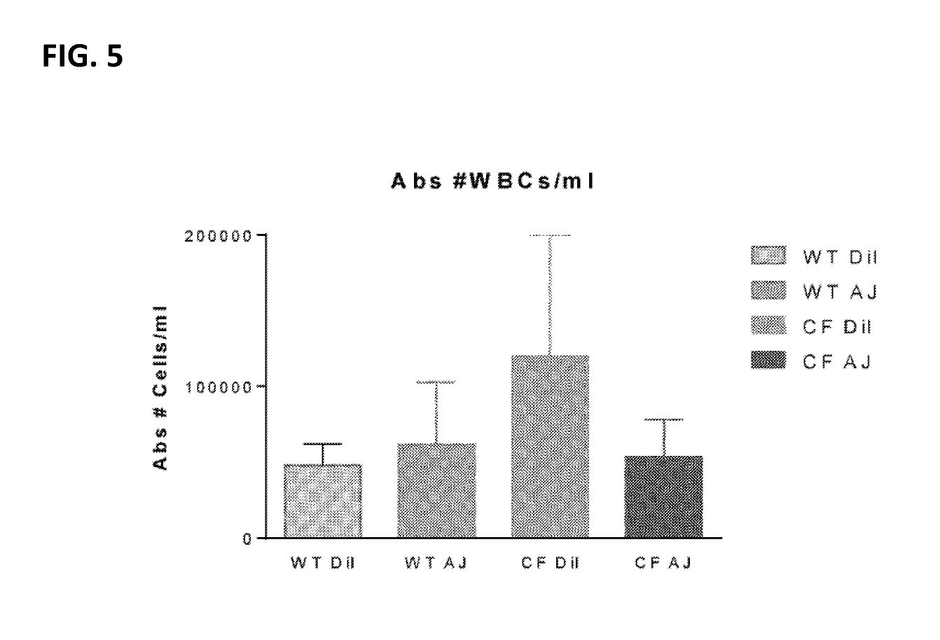

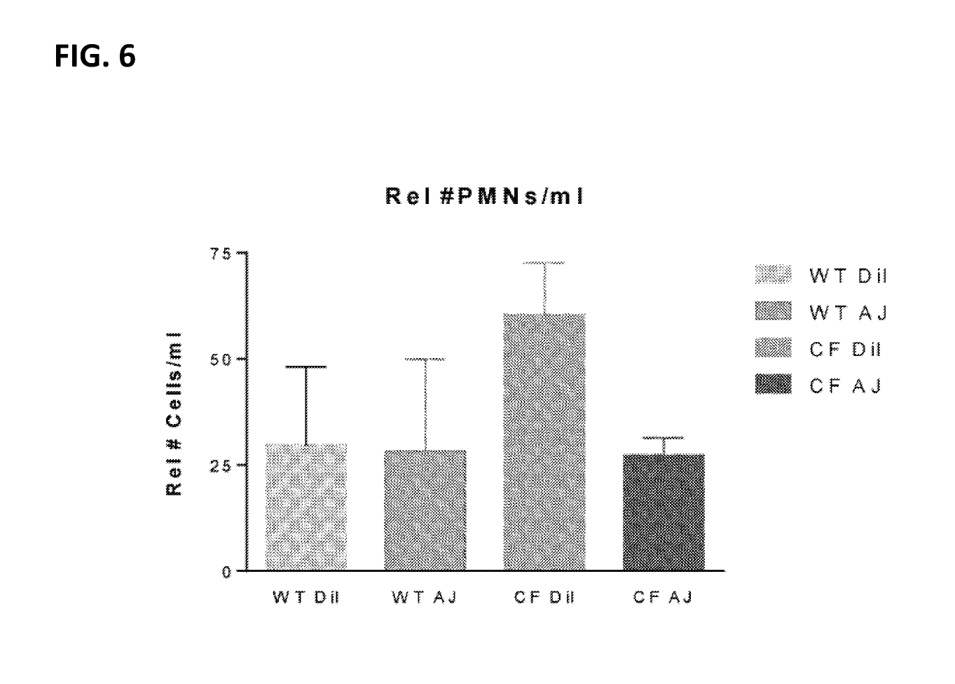

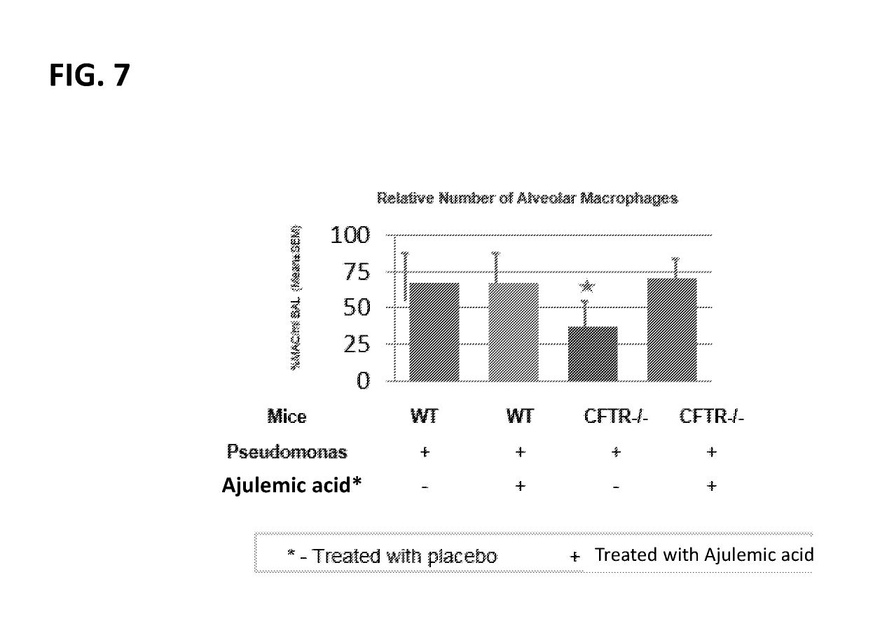

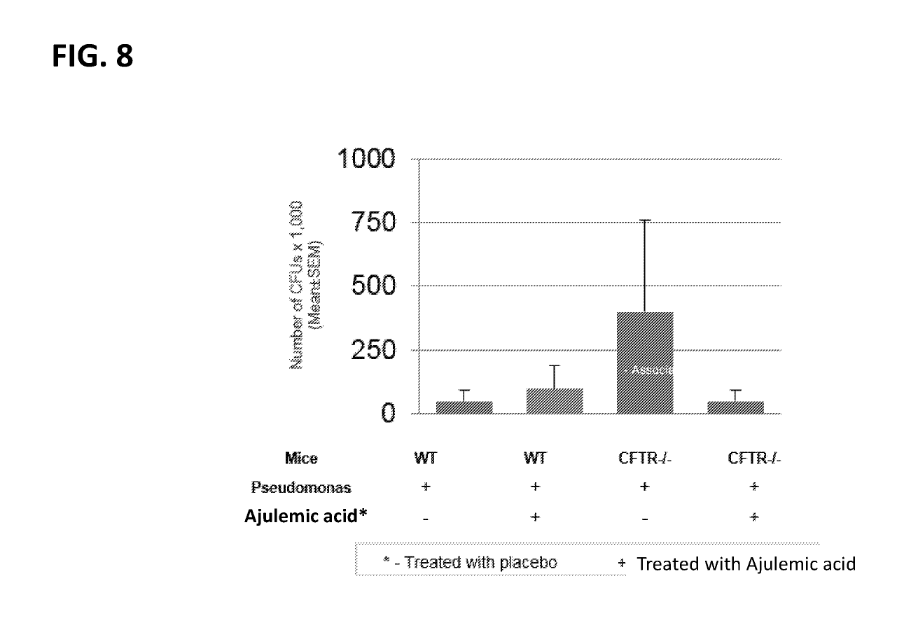

Furthermore, treatment of Cftr KO animals with ajulemic acid decreased weight loss (P<0.01) (FIG. 2), decreased BAL WBC counts (FIGS. 4 and 5), decreased numbers of neutrophils (P<0.05) (FIG. 6), increase the number of alveolar macrophages (FIG. 7), and improved the ability of the animals to resolve pulmonary infection as assessed by lung CFUs (P_{variance}=0.002) (FIG. 8). This study suggests that ajulemic acid may be effective to treat infection in animals having an increased susceptibility to infection and/or a decreased ability to resolve an infection, for example but not limited to, animals having cystic fibrosis.

### Example 3. Study of Resolution of Infection Using a Skin Challenge Model (Also Referred to as the Blister Model)

Summary

The ability of administration of a pharmaceutical composition including ajulemic acid to treat infection was assayed in a skin challenge model. A self-resolving acute inflammatory response was triggered by the intradermal injection of UV-killed Escherichia coli into the forearm of healthy volunteers. While ajulemic acid is known to have anti-inflammatory effects, treatment with ajulemic acid may provide a benefit over alternative anti-inflammatory treatments (e.g., treatment with prednisone or other steroids), which have been shown to reduce bacterial clearance, and therefore the ability to resolve infection. This study was performed to determine whether ajulemic acid promotes the resolution of infection at the site of inflammation, thereby treating that infection.

Cells and exudates were harvested at several time points following injection of UV-killed E. coli by applying negative pressure over the inflamed site. Onset was characterized by high blood flow, neutrophilia, and peak levels of pro-inflammatory cytokines, whilst resolution showed a decline in blood blow, reduction in neutrophils, increase in monocytes/macrophages and waning of classic pro-inflammatory cytokine levels.

Treatment Groups

The study included four experimental groups (n=10 in each group):
1) Placebo, twice daily for four days
2) Ajulemic acid, 5 mg, twice daily for four days
3) Ajulemic acid, 20 mg, twice daily for four days
4) Prednisone, 15 mg daily for four days

The volunteer (healthy males, 18-50 years) were randomly allocated to one of the above three groups and orally administered the test drug for four consecutive days. On the morning of the fourth day (after intake of the first dose), experimental acute inflammation was elicited by infection with UV-killed E. coli. The drug and the placebo were provided as capsules.

Ultraviolet Light Killed E. coli (UVKEc): Preparation and Injection

UV killed E. coli (Strain: NCTC 10418, Source: Public Health England, UK) were prepared as follows. E. coli described were grown overnight in Luria Broth (Sigma) at 37° C. The next morning, the bacteria were washed twice in sterile PBS (2500 g, 20 min, 4° C.) and resuspended in a sterile petri dish. Bacteria were then killed by exposure to an ultraviolet light (UV) source (302 nm, ChemiDoc, trans-UV mode; Bio-Rad laboratories) for 60 min and then washed again in sterile saline. Bacterial counts were determined by optical density (OD600=0.365 equates to 108 E. coli/ml). UVKEc were resuspended in a volume of sterile saline to obtain the count of 1.5×10⁸/ml, aliquoted into sterile eppendorf tubes and then frozen at −80° C. until used for injections.

Intradermal Injection of UVKEc

After disinfecting and shaving the skin, 1.5×10⁷ UVKEc in 100 μl saline were injected intradermally into a marked site on the volar aspect of each forearm. To characterize the treatment of infection using ajulemic acid, each forearm was allotted to one of the predefined time-points namely 4, 8, 14, 24, 48 or 72 hours (h). Thus the effects of intradermal injection of UVKEc were allowed to progress for the duration of the time-point after which a suction blister was raised over the marked injection site, and then aspirated immediately. In summary, volunteer had two injection sites, one on each forearm, and contributed to two time points. On a separate group of volunteers, blister was raised on the naïve skin and treated as the baseline time point. Study time-points were discussed with volunteers before consenting.

Laser Doppler Imaging

Laser Doppler Imager (moor LDI-HIR, Moor Instruments Ltd, Axminster, Devon, UK) was used to quantify the blood flow at the site of infection. At predefined time points after injection of UVKEc, the forearm was placed under the scanner at a fixed distance to scan a fixed area. The scanner emits a laser beam, a portion of which is scattered by red blood cells present at the inflamed area. The scattering causes a change in frequency of the reflected light which is then detected by a photo detector. The velocity and concentration of red blood cells at the site directly affect the Doppler frequency shifts and account for the signal strength measured in arbitrary perfusion units. The data was analysed by moorLDI software (Version 5) and displayed as color coded images showing different blood flow levels over the scanned area. The total blood flow (measured in perfusion units) was calculated as product of number of valid pixels above background signal (Cut-off=300 perfusion units) and the mean blood flow signal over the valid pixels 11, 12.

Induction of Suction Blister

To obtain the exudate from the site of infection, a 10 mm diameter suction blister was induced directly over the site of injection. A suction blister was raised by placing a suction blister chamber connected by tubing to a negative pressure instrument (NP-4, Electronic diversities Ltd., MD, USA). The chamber was made of three parts: an aluminum plate with 10 mm aperture, a nylon cup, and a transparent glass lid, all secured by a detachable air tight seal. The suction chamber was placed on the forearm with the 10 mm aperture centered over the marked injection site. After securely strapping the suction chamber on to the forearm, the negative pressure was applied gradually from 2 to 6-7 inches of Mercury (Hg) until a single uninoculated blister covering the surface area within the aperture was formed. The pressure was brought down gradually to baseline after the blister was completely formed. The suction blister induction process took 1.5-2 h.

Blister Exudate Aspiration

The suction blister was aspirated immediately after formation to collect the exudate. To aspirate the exudate, the blister roof was pierced along its lateral border using a 26.5 gauge needle. The exudate was then gently pushed out onto the skin by rolling a 1 ml syringe over the blister roof and was simultaneously aspirated using a 200 μl pipette tip. The exudate was collected into a well of a 96 well V-bottom plate containing 50 μl of 3% sodium citrate (Sigma) in PBS (Gibco). The plate was then centrifuged at 1000 g for 5 min at 4° C. to separate the cells from the supernatant. After centrifugation, the resulting cell pellet was resuspended in 200 μl of ACK lysis buffer (Lonza) to lyse the red blood cells (RBC). The RBC depleted cell pellet was resuspended in 100 μl of cell staining buffer (PBS with 5% FCS (Gibco)+0.1% sodium azide) and the cell count was obtained using a manual haemocytometer. The supernatant was weighed to estimate the blister fluid volume, split into 30 μl aliquots and then stored at −80° C. The blister area was then cleaned using 0.5% Cetrimide spray (Savlon) and covered with a protective dressing pad (9×10 cm, Mepore).

Peripheral Blood Analysis

Peripheral blood was collected by venopuncture from the medial cubital vein using an aseptic technique. Blood was collected at baseline, 4, 24, 48 and 72 h after UVKEc intradermal injection into EDTA and heparin anti-coagulated vacutainers (BD). For full blood counts, EDTA anti-coagulated blood was sent to an external pathology lab (The Doctor's Laboratory, Whitfield Street, London, UK). Heparin anti-coagulated blood was centrifuged at 2500 g, 10 min, room temperature to separate plasma. Plasma was aliquoted and stored at −80° C. until analysed for cytokines.

Flow Cytometry

Leukocyte subpopulations in the blister fluid were identified by poly-chromatic flow cytometry. For cell surface marker staining, blister cells in 100 μl of cell staining buffer (PBS with 5% FCS+0.1% sodium azide) were incubated with an antibody cocktail. Stained samples were washed in cell wash buffer (PBS with 1% FCS+2 mm EDTA) at 1000 g for 5 min, 4° C. Cells were then fixed in an equal volume of 1% paraformaldehyde and stored in the dark at 4° C. and analysed within 4 h on BD LSR Fortessa™ flow cytometer. Flow cytometry data was analysed by Flowjo software (Treestar Inc.)

Multiplex ELISA

The human cytokine 30-plex kit was purchased from Meso Scale Delivery (MSD, MD, USA). Each kit consists of three 10-plex panels—Proinflammatory Panel 1, Cytokine Panel 1 and Chemokine Panel 1. The supernatant from blister exudate or the plasma was diluted in appropriate assay diluent and the assay was performed as per manufacturer's instructions. All assay components were supplied by the manufacturer.

Summary of Results from Blister Model

UV-killed E. coli (UVKEc) were injected subcutaneously to induce an innate immune response in humans. Healthy male volunteers were randomized to receive either Placebo, 5 mg AJA BID, 20 mg AJA BID, or 15 mg prednisone QD for four days. On fourth day, acute inflammation was triggered by intradermal injection of UV killed E. coli on both the forearms. Blisters were induce at 4 hours or 10 hours post-injection to collect and evaluate the levels of lipid mediators and cells.

Ajulemic acid was found to reduce vasodilation, Chemokine IL-8 production, and tissue infiltration with neutrophils. Results are for the treatment on inflammation are similar magnitude to that resulting for treatment with corticosteroids (e.g., prednisone). Importantly, treatment with prednisone does not increase bacterial clearance and may slow the rate of bacterial clearance, whereas, treatment with ajulemic acid decreased levels of endotoxin at the site of injection suggesting that ajulemic acid may be effective at increasing bacterial clearance and thereby treating infection.

Ajulemic Acid Treatment May Increase Local Blood Flow

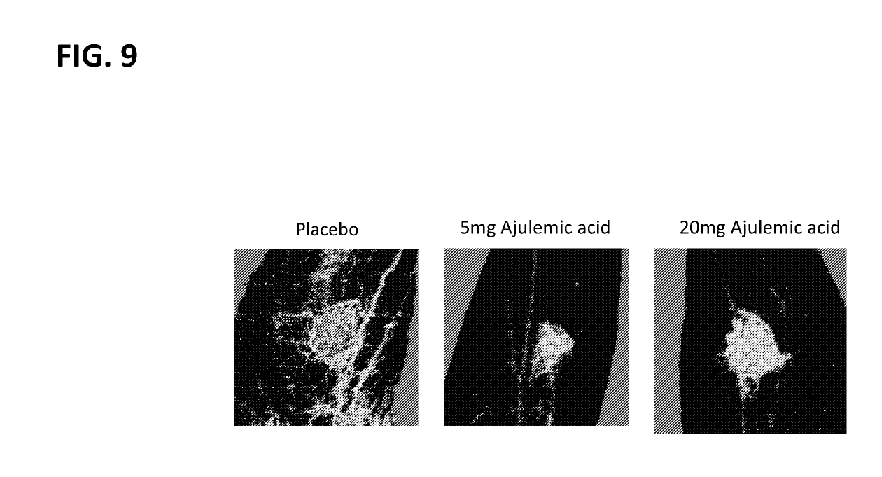

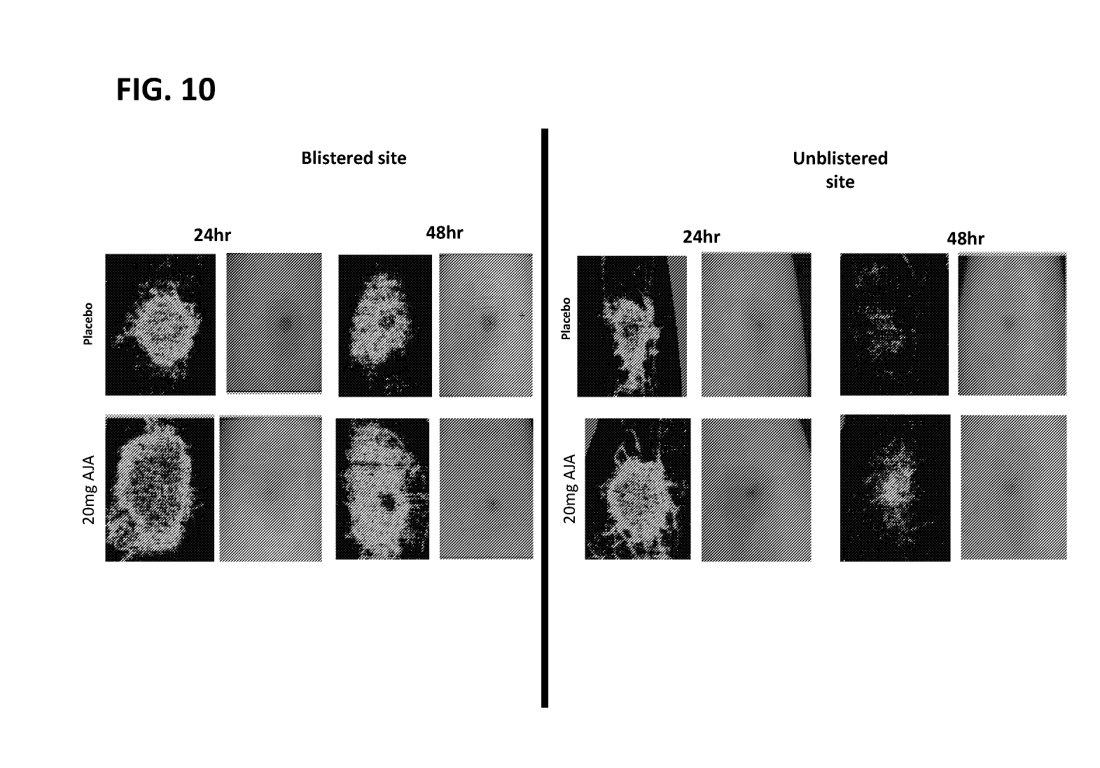

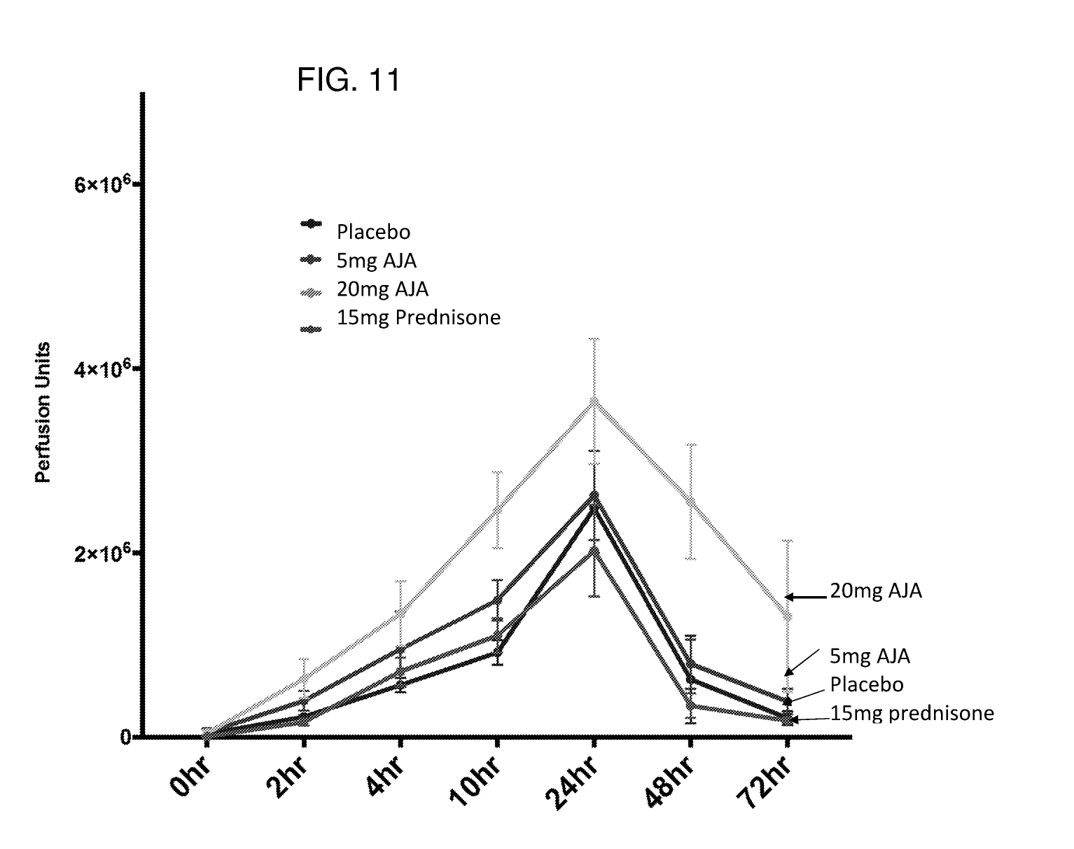

Vascular hyperaemia was observed at the site of UVkEc triggered inflammation after treatment with placebo, 5 mg AJA, 20 mg AJA, and 15 mg prednisone (FIGS. 9-11). Total blood flow at the injection site was assessed at specified time points by a laser Doppler imager (moorLDI-HIR). The images an corresponding quantification of local vascular blood flow show an increase in local blood flow at, at least, 20 mg AJA, which suggests that 20 mg AJA may be triggering a potent pro-resolution factor.

Ajulemic Acid Treatment May Decrease Neutrophil Infiltration

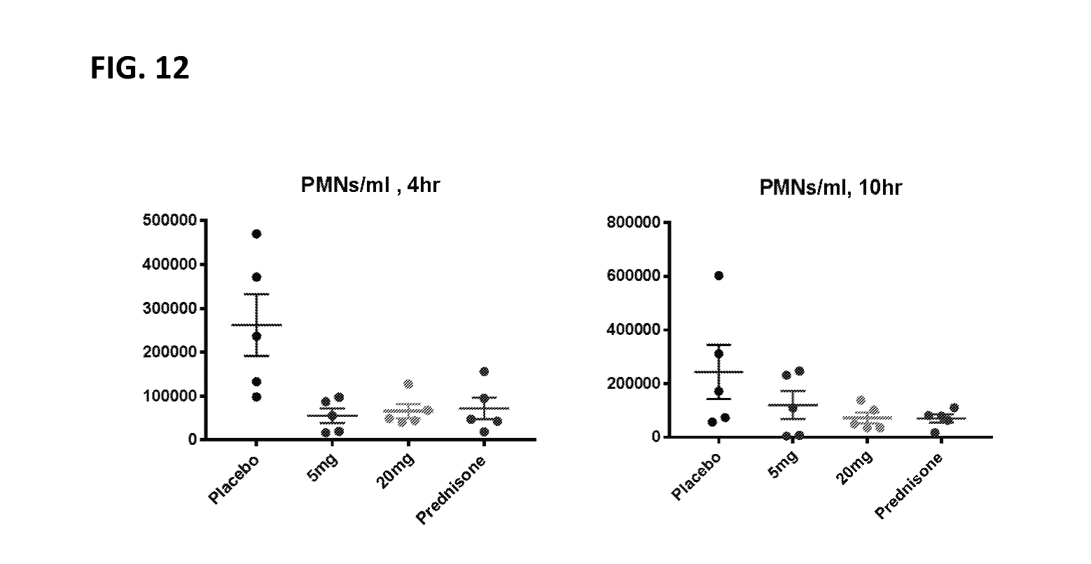

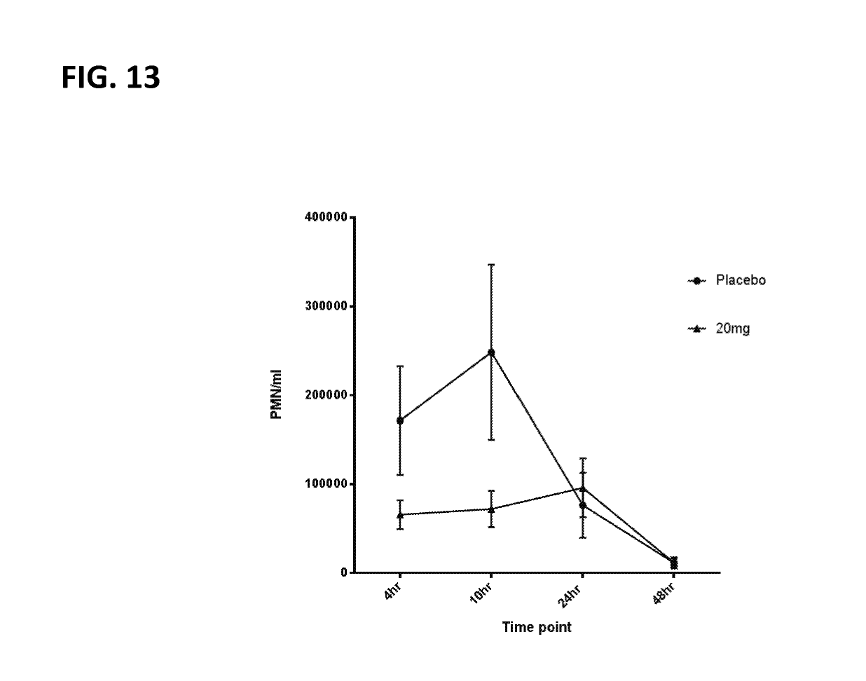

Inflammatory exudate at the injection site was acquired into a suction blister raised after 4 h (onset phase) on one forearm and after 10 h (resolution phase) on the contralateral forearm. Neutrophils in the exudate were phenotyped by multicolor flow cytometry as (HLA-DR−/CD16++). FIG. 12 shows a decrease in the infiltration of neutrophils at the site of inflammation following treatment with ajulemic acid or prednisone, relative to placebo. FIG. 13 shows a time course of neutrophil infiltration at the site of inflammation in the 20 mg ajulemic acid group, and again, neutrophil infiltration is decreased relative to placebo. Therefore, while ajulemic acid appears to increase blood flow at the site of infection, it does not appear to cause an influx of neutrophils (e.g., polymorphonuclear neutrophils or PMNs).

Ajulemic Acid Treatment May Increase in Mononuclear Phagocytes (Macrophages)

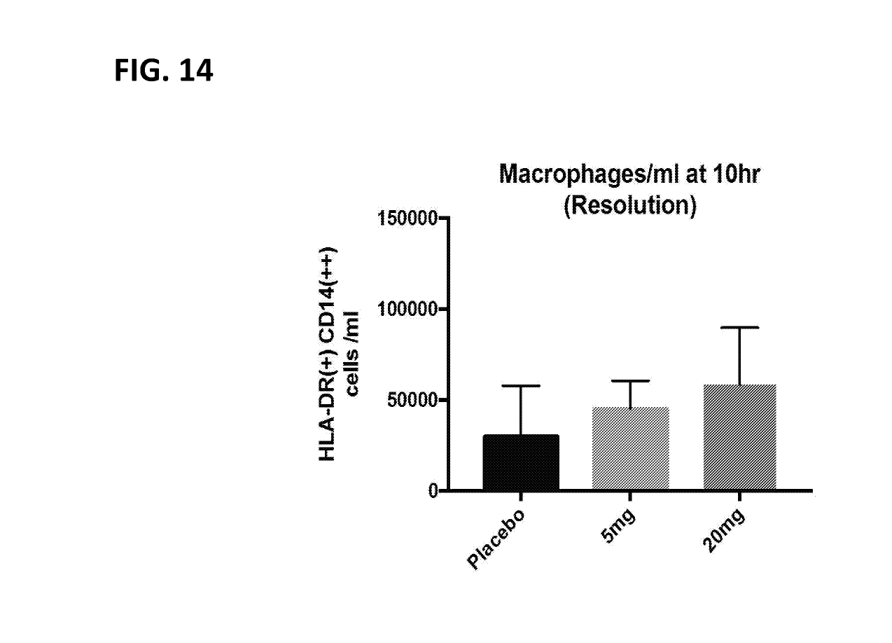

Inflammatory exudate at the injection site was acquired into a suction blister raised after 4 h (onset phase) on one forearm and after 10 h (resolution phase) on the contralateral forearm. Monocytes/Macrophages in the exudate were phenotyped by multi-colour flow cytometry as HLA-DR⁺ CD14⁺⁺ cells. FIG. 14 shows that treatment with ajulemic acid may increase macrophages infiltration at the site of injection of UVKEc.

Treatment with Ajulemic Acid May Increase CD163 and CD86 Expression on Monocytes/Macrophages

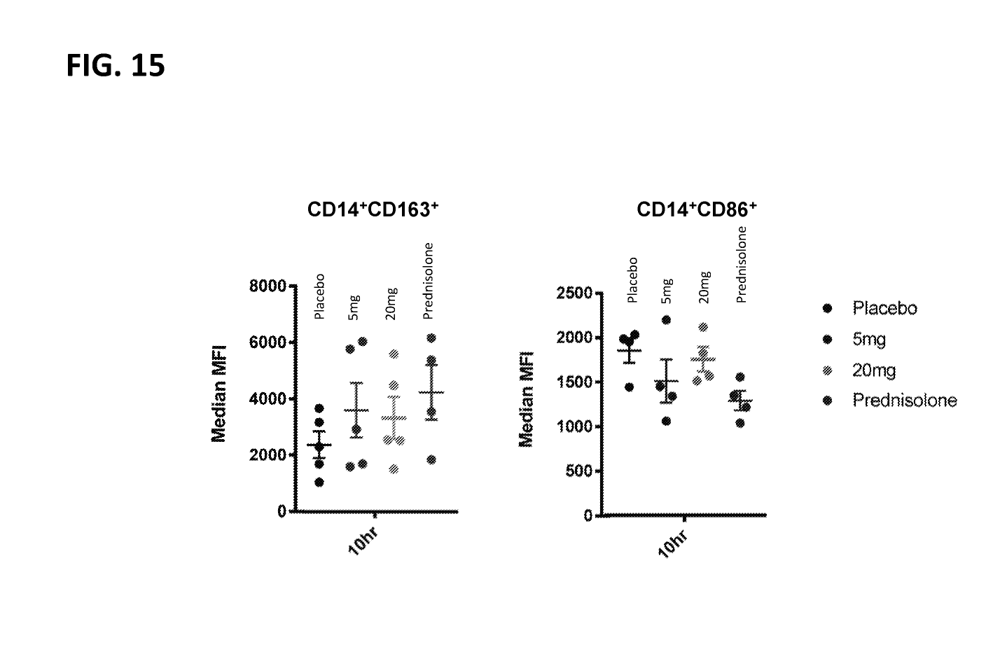

Inflammatory exudate at the injection site was acquired into a suction blister raised after 4 h (onset phase) on one forearm and after 10 h (resolution phase) on the contralateral forearm. Monocyte/macrophage in the exudate were phenotyped by multi-color flow cytometry. The surface expression (median fluorescence intensity-MFI) of CD163 and CD86 monocytes/macrophages at 4 hr and 10 hr are shown in FIG. 15. The data suggests that ajulemic acid treatment may cause an increase in CD163 and CD86 expression on monocytes/macrophages.

Treatment with Ajulemic Acid May Reduce Levels of Pro-Inflammatory Cytokines

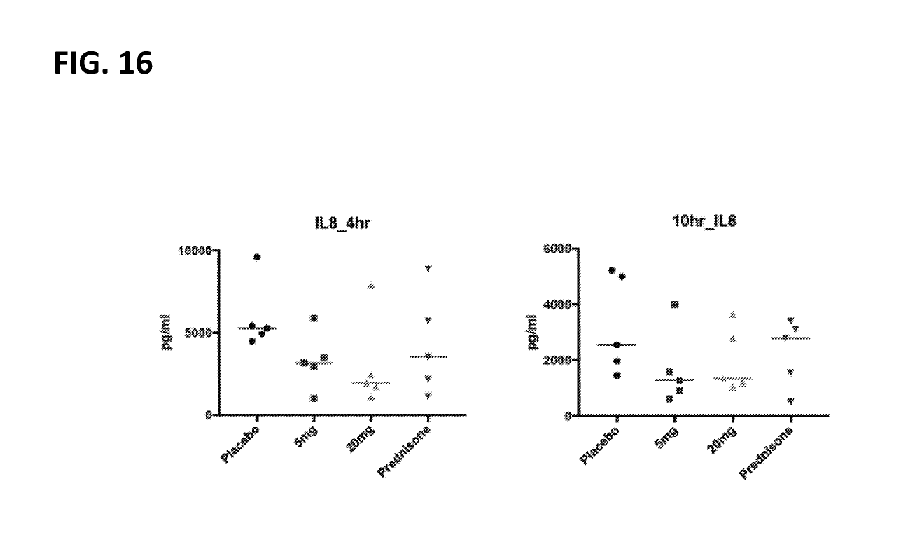

Inflammatory exudate at the injection site was acquired into a suction blister raised after 4 h (onset phase) on one forearm and after 10 h (resolution phase) on the contralateral forearm. IL-8 cytokine in the inflammatory exudate was measured using multiplex ELISA (MSD). FIG. 16 shows that treatment with ajulemic acid may reduce levels of pro-inflammatory cytokines, such as IL-8.

Treatment with Ajulemic Acid May Reduce Levels of Endotoxin, which Suggests Increased Bacterial Clearance at the Site of UVKEc Injection

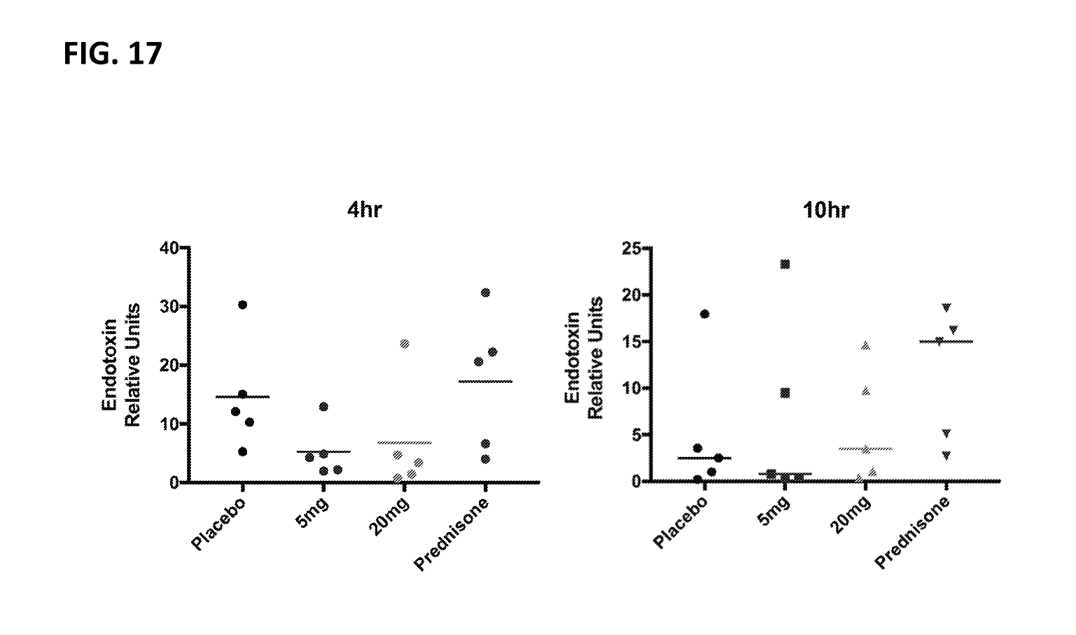

Inflammatory exudate at the injection site was acquired into a suction blister raised after 4 h (onset phase) on one forearm and after 10 h (resolution phase) on the contralateral forearm. Endotoxin was measured using kinetic turbidimetric limulus ameobocyte lysate test. As shown in FIG. 17, treatment with ajulemic acid reduces levels of endotoxin in the model of intradermal UV-killed E. coli-driven inflammation in humans. This suggests an increase in bacterial clearance at the site of injection. Notably, no such decrease in bacterial clearance is observed following treatment with prednisone. In fact, prednisone treatment appears to increase the levels of endotoxin suggesting a decrease in the rate of bacterial clearance at the site of injection.

Treatment with Ajulemic Acid does not Inhibit Rise in C-Reactive Protein (CRP)

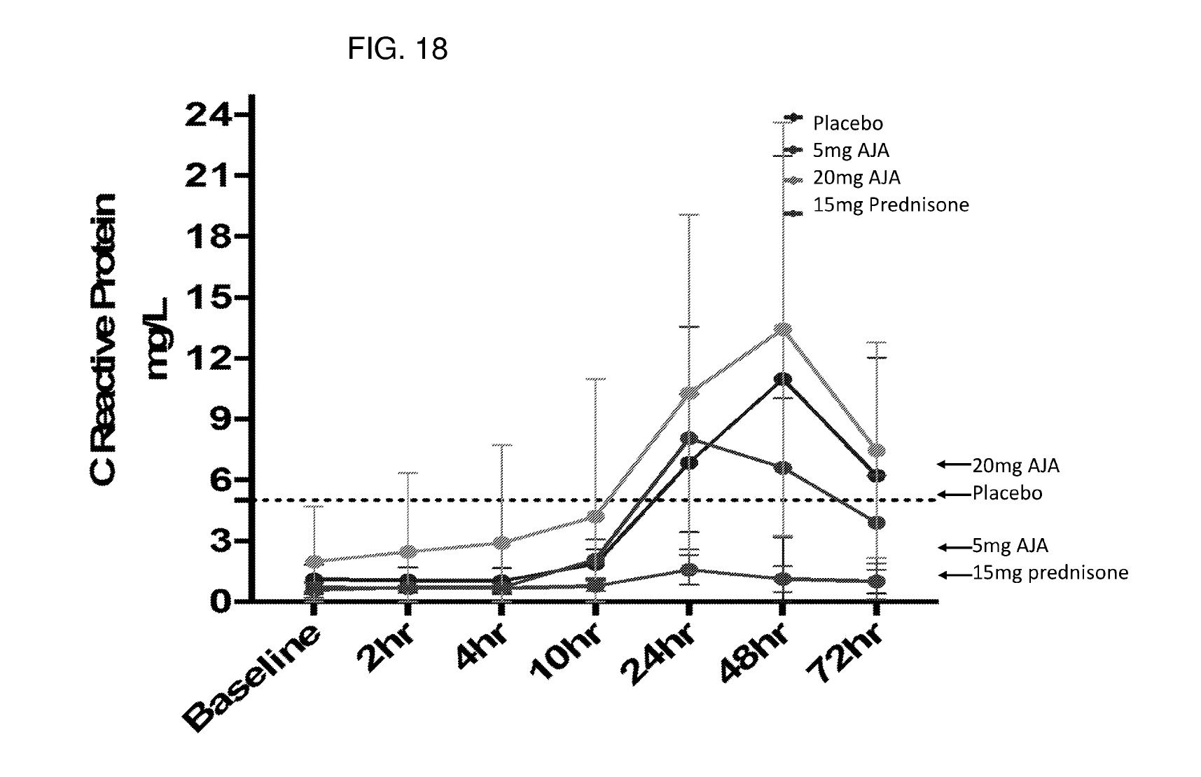

Peripheral blood was taken at multiple time points after inflammation and the serum prepared from it was analysed for C-reactive protein (CRP) (The Doctor's laboratory, London, UK). FIG. 18 shows that treatment with ajulemic acid does not appear to inhibit the rise in C-reactive protein levels in the serum after UVkEc triggered acute inflammation. This is notable since CRP promotes opsonization (e.g., the immune process where particles such as bacteria are targeted for destruction by phagocytes). By contrast, prednisone does appear to inhibit the rise in CRP.

### Example 4. Ajulemic Acid (Anabasum) Reduces Acute Pulmonary Exacerbations in a Phase 2 Study of Subjects Having Cystic Fibrosis

Administration of a pharmaceutical composition including ajulemic acid was evaluated for its ability to reduce acute pulmonary exacerbations in a Phase 2 study of subjects having cystic fibrosis. Treatment with ajulemic acid reduced acute pulmonary exacerbations requiring treatment with intravenous antibiotics compared to the placebo arm. Treatment with ajulemic acid also reduced acute pulmonary exacerbations requiring treatment with new antibiotics compared to the placebo arm. A reduction was observed in all treatment groups, with the greatest reduction observed in subjects on the highest dose (20 mg, twice a day).

Study Design

An international, multi-center, double-blinded, randomized, placebo-controlled Phase 2 study was performed in subjects having cystic fibrosis. The primary objective of the study was to test safety and tolerability of ajulemic acid in adults with cystic fibrosis who had forced expiratory volume in 1 second (FEV1) percent predicted at least 40% predicted, without regard to their CFTR mutation, infecting pathogen, or baseline treatment. Events of special interest included acute exacerbations requiring intravenous antibiotic treatment or treatment with new antibiotics.

Eighty-five subjects on stable standard-of-care medications were dosed with ajulemic acid at 21 cystic fibrosis centers in the U.S. and Europe and treated with ajulemic acid daily for a period of 84 days, with a follow-up period of 28 days. During the first part of the study (weeks 1-4) subjects were randomized to the following treatment groups: placebo (n=35), 1 mg/day ajulemic acid (n=26) or 5 mg/day ajulemic acid (n=24). During the second part of the study (weeks 5-13), subjects given ajulemic acid in the first part of the study were re-randomized to ajulemic acid 20 mg once per day (n=31) or ajulemic acid 20 mg twice a day (n=30) with 11 subjects from the placebo group also being randomized to the ajulemic acid arms. The last part of the study was a 28 day follow up period post treatment.

Results

Eighty-five subjects were dosed with study drug of which 74 completed the study. Three subjects withdrew consent, 5 withdrew due to adverse events (2 on placebo, 3 on anabasum), 1 subject was lost to follow-up, and 2 subjects withdrew for treatment-unrelated reasons. Baseline characteristics were overall similar between ajulemic acid and placebo arms. No related serious or severe treatment emergent adverse events were observed.

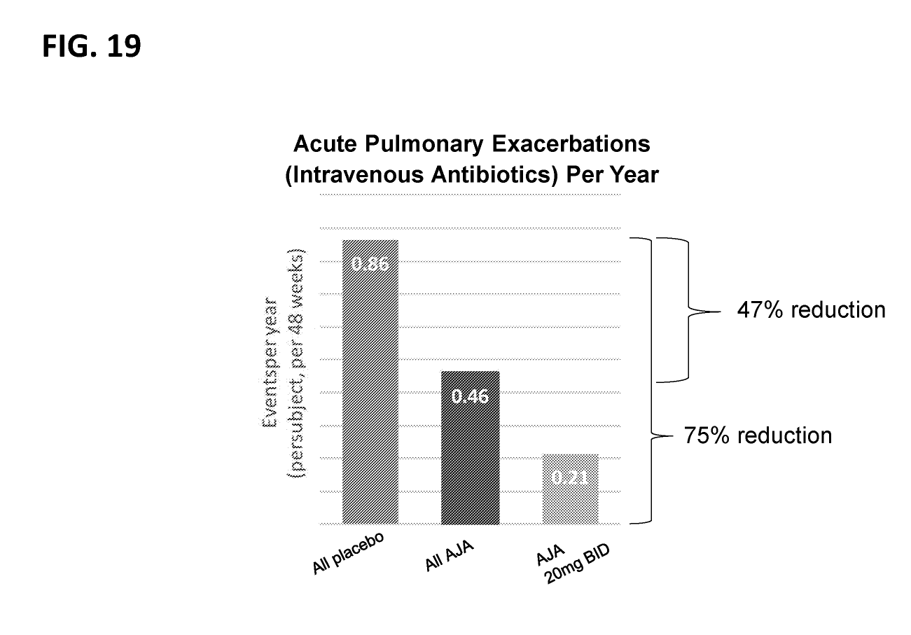

Treatment with ajulemic acid resulted in a dose-dependent decrease in the occurrence of acute pulmonary exacerbations requiring treatment with intravenous antibiotics when compared to placebo (FIG. 19). A 75% reduction was observed in the 48 week rate of acute pulmonary exacerbations in subjects treated with ajulemic acid at 20 mg BID. Reductions were also observed in subjects administered 1 mg/day, 5 mg/day, or 20 mg/day of ajulemic acid for their respective treatment periods. A summary of the observed occurrence of pulmonary exacerbations requiring treatment with intravenous antibiotics is provided in Table 3.

TABLE 3
Acute pulmonary exacerbations requiring intravenous antibiotics

Subjects, n (% at risk)

| Treatment Group | Weeks 1-4 | Weeks 5-12 |
|---|---|---|
| Placebo | 3 (8.6) | |
| Ajulemic acid 1 mg | 1 (3.8) | |
| Ajulemic acid 5 mg | 1 (4.2) | |
| Placebo | | 3 (16.7) |
| Ajulemic acid 20 mg | | 3 (6.5) |
| Ajulemic acid 20 mg BID | | 1 (3.3) |

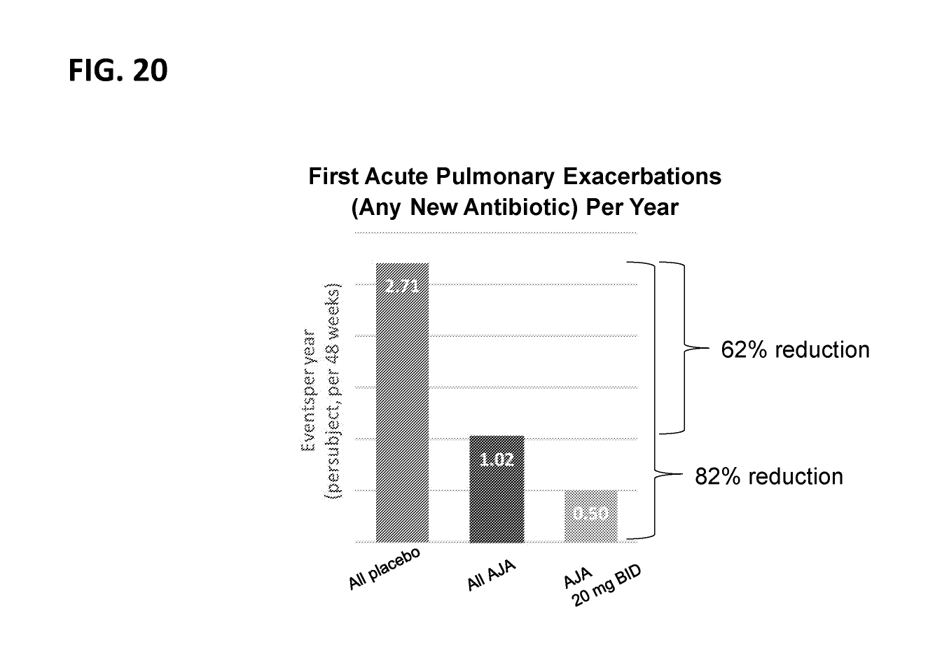

Treatment with ajulemic acid also resulted in a dose-dependent decrease in the occurrence of acute pulmonary exacerbations requiring treatment with any new antibiotic (e.g., subjects who were being treated chronically with one or more antibiotics, who further required treatment with a new antibiotic due to an acute pulmonary exacerbation) (FIG. 20). An 82% reduction was observed in the 48 week rate of acute pulmonary exacerbations in subjects treated with ajulemic acid at 20 mg BID. Reductions were also observed in subjects administered 1 mg/day, 5 mg/day, or 20 mg/day of ajulemic acid for their respective treatment periods. A summary of the observed occurrence of pulmonary exacerbations requiring treatment with a new antibiotic is provided in Table 4.

TABLE 4
Acute pulmonary exacerbations requiring a new antibiotic

| | Subjects n/N at risk (%) | | |
|---|---|---|---|
| Treatment Group, N at risk for 1st | | | Post- |
| exacerbation | Weeks 1-4 | Weeks 5-12 | treatment |
| Placebo, N = 34 | 6 (25.0) | | |
| Ajulemic acid 1 mg, N = 22 | 3 (13.6) | | |
| Ajulemic acid 5 mg, N = 23 | 3 (13.0) | | |
| Placebo, N = 18 | | 9 (50.0) | |
| Ajulemic acid 20 mg, N = 25 | | 4 (16.0) | |
| Ajulemic acid 20 mg BID, N = 24 | | 2 (8.3) | |
| Placebo, N = 9 | | | 2 (22.2) |
| Ajulemic acid 20 mg, N = 20 | | | 6 (30.0) |
| Ajulemic acid 20 mg BID, N = 22 | | | 2 (9.1) |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of treating an infection in a subject in need thereof, the method comprising administering to said subject a pharmaceutical composition comprising ajulemic acid, or a pharmaceutically acceptable salt thereof, in an amount effective to treat said infection, wherein said subject does not have cystic fibrosis or an HIV infection.

2. A method of treating a local infection in a subject in need thereof, the method comprising administering to said subject a pharmaceutical composition comprising ajulemic acid, or a pharmaceutically acceptable salt thereof, in an amount effective to treat said local infection, wherein said subject does not have cystic fibrosis.

3. The method of claim 2, wherein said local infection is a skin infection, a lung infection, a bronchial infection, a throat infection, an eye infection, an ear infection, a bladder infection, or a urinary tract infection.

4. A method of treating a systemic infection in a subject in need thereof, the method comprising administering to said subject a pharmaceutical composition comprising ajulemic acid, or a pharmaceutically acceptable salt thereof, in an amount effective to treat said systemic infection, wherein said subject does not have an HIV infection.

5. The method of anyone of claims 1-4, wherein the infection is a bacterial infection.

6. The method of 5, wherein the bacterial infection is a pseudomonas, staphylococcus, or streptococcus infection.

7. The method of claim 5 or 6, wherein administration of the pharmaceutical composition comprising ajulemic acid reduces the bacterial burden of the infection.

8. The method of anyone of claims 1-4, wherein the infection is a viral infection.

9. The method of claim 8, wherein administration of the pharmaceutical composition comprising ajulemic acid reduces the viral load of the infection.

10. The method of anyone of claims 1-4, wherein the infection is a fungal infection.

11. The method of claim 10, wherein administration of the pharmaceutical composition comprising ajulemic acid reduces the fungal load of the infection.

12. A method of treating a bacterial infection in a subject in need thereof, the method comprising administering to said subject a pharmaceutical composition comprising ajulemic acid, or a pharmaceutically acceptable salt thereof, in an amount effective to treat said bacterial infection.

13. The method of 12, wherein the bacterial infection is a pseudomonas, staphylococcus, or streptococcus infection.

14. The method of claim 12 or 13, wherein administration of the pharmaceutical composition comprising ajulemic acid reduces the bacterial burden of the infection.

15. A method of treating a viral infection in a subject in need thereof, the method comprising administering to said subject a pharmaceutical composition comprising ajulemic acid, or a pharmaceutically acceptable salt thereof, in an amount effective to treat said viral infection.

16. The method of claim 15, wherein administration of the pharmaceutical composition comprising ajulemic acid reduces the viral load of the infection.

17. A method of treating a fungal infection in a subject in need thereof, the method comprising administering to said subject a pharmaceutical composition comprising ajulemic acid, or a pharmaceutically acceptable salt thereof, in an amount effective to treat said fungal infection.

18. The method of claim 17, wherein administration of the pharmaceutical composition comprising ajulemic acid reduces the fungal load of the infection.

19. A method of treating a bacterial infection in a subject in need thereof, the method comprising the steps of:
(e) administering a pharmaceutical composition comprising an antibiotic, or a pharmaceutically acceptable salt thereof; and
(f) administering a pharmaceutical composition comprising ajulemic acid, or a pharmaceutically acceptable salt thereof;
wherein the length of time associated with resolution of the bacterial infection is less than the length of time associated with resolution of a bacterial infection of the same type in a subject who has been administered the pharmaceutical composition comprising the antibiotic alone.

20. A method of treating a viral infection in a subject in need thereof, the method comprising the steps of:
(c) administering a pharmaceutical composition comprising an antiviral, or a pharmaceutically acceptable salt thereof; and
(d) administering a pharmaceutical composition comprising ajulemic acid, or a pharmaceutically acceptable salt thereof;
wherein the length of time associated with resolution of the viral infection is less than the length of time associated with resolution of a viral infection of the same type in a subject who has been administered the pharmaceutical composition comprising the antiviral alone.

21. A method of treating a fungal infection in a subject in need thereof, the method comprising the steps of:
(e) administering a pharmaceutical composition comprising an antifungal, or a pharmaceutically acceptable salt thereof; and
(f) administering a pharmaceutical composition comprising ajulemic acid, or a pharmaceutically acceptable salt thereof;
wherein the length of time associated with resolution of the fungal infection is less than the length of time associated with resolution of a fungal infection of the same type in a subject who has been administered the pharmaceutical composition comprising the antifungal alone.

22. The method of claims 19-21, wherein the step (a) is performed for a first period of time, step (b) is performed for a second period of time, and step (a) precedes step (b).

23. The method of claims 19-21, wherein the step (b) is performed for a first period of time, step (a) is performed for a second period of time, and step (b) precedes step (a).

24. The method of claims 19-21, wherein the step (a) is performed for a first period of time, step (b) is performed for a second period of time, and the first period of time and the second period of time occur concurrently.

25. A method of treating a bacterial infection in a subject in need thereof, the method comprising administering to said subject a pharmaceutical composition comprising an antibiotic, or a pharmaceutically acceptable salt thereof, and ajulemic acid, or a pharmaceutically acceptable salt thereof, in an amount effective to treat said bacterial infection.

26. The method of claim 25, wherein the bacterial infection is a pseudomonas, staphylococcus, or streptococcus infection.

27. The method of claim 25 or 26, wherein administration of the pharmaceutical composition comprising ajulemic acid, or a pharmaceutically acceptable salt thereof, reduces the bacterial burden of the infection.

28. A method of treating a fungal infection in a subject in need thereof, the method comprising administering to said subject a pharmaceutical composition comprising an antifungal, or a pharmaceutically acceptable salt thereof, and ajulemic acid, or a pharmaceutically acceptable salt thereof, in an amount effective to treat said fungal infection.

29. The method of claim 28, wherein administration of the pharmaceutical composition comprising ajulemic acid, or a pharmaceutically acceptable salt thereof, reduces the fungal load of the infection.

30. A method of treating a viral infection in a subject in need thereof, the method comprising administering to said subject a pharmaceutical composition comprising an antiviral, or a pharmaceutically acceptable salt thereof, and ajulemic acid, or a pharmaceutically acceptable salt thereof, in an amount effective to treat said viral infection.

31. The method of claim 30, wherein administration of the pharmaceutical composition comprising ajulemic acid, or a pharmaceutically acceptable salt thereof, reduces the viral load of the infection.

32. The method of anyone of claims 1-31, wherein the length of time associated with resolution of the infection is decreased by 20% or more, as compared to an infection of the same type in a subject who has not been administered the pharmaceutical composition comprising ajulemic acid, or a pharmaceutically acceptable salt thereof.

33. The method of anyone of claims 1-31, wherein the length of time associated with resolution of the infection is decreased by 50% or more, as compared to an infection of the same type in a subject who has not been administered the pharmaceutical composition comprising ajulemic acid, or a pharmaceutically acceptable salt thereof.

34. The method of anyone of claims 1-33, wherein the pharmaceutical composition comprising ajulemic acid, or a pharmaceutically acceptable salt thereof, is administered orally, by inhalation, topically, intravenously, interstitially, via a patch, via an implant, or by ophthalmic administration.

35. The method of claim 34, wherein the pharmaceutical composition comprising ajulemic acid, or a pharmaceutically acceptable salt thereof, is administered orally.

36. The method of claim 35, wherein the pharmaceutical composition comprising ajulemic acid, or a pharmaceutically acceptable salt thereof, is administered as a capsule or a tablet.

37. The method of claim 35 or 36, wherein the effective amount of ajulemic acid, or a pharmaceutically acceptable salt thereof, comprises a dose of about 5 mg per day.

38. The method of claim 35 or 36, wherein the effective amount of ajulemic acid, or a pharmaceutically acceptable salt thereof, comprises a dose of about 20 mg per day.

39. The method of claim 35 or 36, wherein the effective amount of ajulemic acid, or a pharmaceutically acceptable salt thereof, comprises a dose of about 40 mg per day.

40. The method of claim 34, wherein the pharmaceutical composition comprising ajulemic acid, or a pharmaceutically acceptable salt thereof, is administered by inhalation.

41. The method of claim 40, wherein the pharmaceutical composition comprising ajulemic acid, or a pharmaceutically acceptable salt thereof, is administered as an aerosol or a spray.

42. The method of claim 34, wherein the pharmaceutical composition comprising ajulemic acid, or a pharmaceutically acceptable salt thereof, is administered topically.

43. The method of claim 42, wherein the pharmaceutical composition comprising ajulemic acid, or a pharmaceutically acceptable salt thereof, is administered as a gel or a cream.

44. The method of anyone of claims 1-43, wherein the subject is a human subject.

45. The method of anyone of claim 4-44, wherein the subject does not have cystic fibrosis.

46. The method anyone of claims 2-45, wherein the subject does not have HIV.
